(19) United States
(12) Patent Application Publication
(10) Pub. No.: US 2025/0145939 A1
(43) Pub. Date: May 8, 2025

(54) PAENIBACILLUS STRAINS PRODUCING LOW AMOUNTS OF EXOPOLYSACCARIDES

(71) Applicant: BASF SE, Ludwigshafen (DE)

(72) Inventors: Tobias May, Neustadt An Der Weinstrasse (DE); Daniel Christoph Heinrich, Ludwigshafen (DE); Andrea Herold, Ludwigshafen (DE); Reinhard Stierl, Limburgerhof (DE)

(21) Appl. No.: 18/684,437

(22) PCT Filed: Aug. 9, 2022
(86) PCT No.: PCT/EP2022/072287

§ 371 Date: Feb. 16, 2024

(30) Foreign Application Priority Data
Aug. 20, 2021 (EP) 21192437.8

(51) Int. Cl. C12N 1/20; A01N 63/22; A01P 3/00; A01P 21/00; C07K 14/32; C12N 9/10; C12N 9/12; C12P 7/18; C12P 7/26; C12P 7/56; C12R 1/07

(52) U.S. Cl. CPC C12N 1/20; A01N 63/22; A01P 3/00; A01P 21/00; C07K 14/32; C12N 9/1055; C12N 9/1241; C12P 7/18; C12P 7/26; C12P 7/56; C12R 2001/07; C12Y 204/0101; C12Y 207/07013

(57) ABSTRACT

Disclosed are Paenibacillus strains comprising a reduced activity of a flippase PepR aflippase PepH, a mannose-1-phosphate guanylyl transferase and/or a levansucrase SacB. These Paenibacillus strains show lower viscosity when grown in liquid culture and agricultural compositions comprising these strains and methods of making and using these strains.

### FIELD

The present invention relates to Paenibacillus strains comprising a reduced activity of a flippase PepR, a flippase PepH, a mannose-1-phosphate guanylyl transferase and/or a levansucrase SacB. These Paenibacillus strains show lower viscosity when grown in liquid culture. The present invention relates also to compositions comprising these strains and methods making use of these strains.

### BACKGROUND

Paenibacillus is known for its capability to produce several kinds of exopolysaccharides (EPS) (Liang and Wang, Recent Advances in Exopolysaccharides from Paenibacillus spp.: Production, Isolation, Structure, and Bioactivities, Mar. Drugs 2015, 13, 1847 to 1863). The main components of EPS produced by Paenibacillus species described so far are:
1) levan, a polymer of fructose linked by a β-(2→6) fructofuranosidic bonds, (Bezzate et al, Disruption of the Paenibacillus polymyxa levansucrase gene impairs its ability to aggregate soil in the wheat rhizosphere, Environmental Microbiology 2000 2(3), 333 to 342),
2) curdlan, a linear glucan composed entirely of (1→3)-d-glycosidic linkages, which forms coaxial triple helixes, (Rafigh et al., Optimization of culture medium and modeling of curdlan production from Paenibacillus polymyxa by RSM and ANN, International Journal of Biological Macromolecules 70 (2014) 463-473) and
3) paenan, a heteropolysaccharide comprising glucose, mannose, galactose, and glucuronic acid (Marius Rütering, Exopolysaccharides by Paenibacilli: from Genetic Strain Engineering to Industrial Application, Dissertation, 2019).

Paenibacilli use several pathways to produce these EPS. In addition to the gene for levansucrase (SacB) required for the production of levan they are carrying several gene clusters. One known gene cluster comprises 29 genes for the production of EPS. (Marius Rütering, Exopolysaccharides by Paenibacilli: from Genetic Strain Engineering to Industrial Application, Dissertation, 2019). In addition to genes for the production of the building blocks of the EPS and genes encoding functions for the transport of the EPS trough the peptidoglycan cell wall, this gene cluster comprises two flippases (pepH and pepR), which provide the function to transfer lipid bound oligosaccharides trough the cell membrane and two polymerases (pepE and pepG) to polymerize the transported oligosaccharides to form the final EPS (Marius Rütering, Exopolysaccharides by Paenibacilli: from Genetic Strain Engineering to Industrial Application, Dissertation, 2019). The exact mechanism to produce curdlan in Paenibacilli is still unknown. However, it is assumed that paenan is produced via a membrane located synthetase (Marius Rütering, Exopolysaccharides by Paenibacilli: from Genetic Strain Engineering to Industrial Application, Dissertation, 2019).

While the produced EPS fulfill important functions for Paenibacilli in natural environments, like the formation of biofilms, they tend to be produced in excess amounts when rich supplies of sugars are present, like in artificial growth conditions in industrial production processes. This excess production of EPS results in highly viscous culture broths, which are difficult to stir and cause problems to keep all areas of the fermenter optimally aerated and equally provided with nutrients. In cases in which the aim of the industrial process is not the production of EPS, its excess production does not only represent a metabolic burden and a sink of valuable nutrients, but frequently causes additional problems in down-stream-processing and isolation of the intended product of economic value, such as spores and biomass of Paenibacilli, enzymes, secondary metabolite compounds or low weight chemicals, like 2,3-Butanediol.

In order to avoid such problems several approaches have been taken to reduce the capacity of Paenibacilli to produce EPS. These approaches usually used deletions of individual genes or deletion of large portions of EPS production clusters to destroy the ability to produce one or several EPS. For example: Okonkwo et al. Inactivation of the Levansucrase Gene in Paenibacillus polymyxa DSM 365 Diminishes Exopolysaccharide Biosynthesis during 2,3-Butanediol Fermentation, Applied and Environmental Microbiology, Volume 86 Issue 9, 2020, e00196-20 deleted the gene encoding for levansucrase. These approaches included also the deletion of the levansucrase gene and additional 16 genes of the EPS gene cluster of Paenibacillus, including both genes for the EPS polymerases (pepE and pepG) and one gene of the two flippase genes (pepH). The same gene cluster has been examined in He et al. Effects of an EPS Biosynthesis Gene Cluster of Paenibacillus polymyxa WLY78 on Biofilm Formation and Nitrogen Fixation under Aerobic Conditions, Microorganisms 2021, 9, 289. https://doi.org/10.3390/microorganisms9020289. He et al. deleted the whole gene cluster in two separate large deletions and found that a large deletion comprising pepH resulted in strains producing a low amount of EPS, but found that deletion of the second half of the large gene cluster comprising pepR had no influence on the production of EPS.

While these approaches have been successful in reducing the EPS production during large scale fermentation, there still remains a need for less drastic approaches, which have a less severe effect on the organism's metabolism, but nevertheless reduce the EPS production during large scale fermentation, in particular when high concentrations of glucose and/or saccharose are present in the growth medium. There is also a need for new strains which show a sufficiently reduced EPS production during large scale fermentation but keeping the ability to produce sufficient amounts of EPS to allow for biofilm formation and interaction with plants when grown under natural conditions.

Surprisingly, it has been found that reducing the activity of one flippase of Paenibacilli is sufficient to create strong effects on the total EPS production. Similar surprising it has been found that a mutant levansucrase, can lead to a stronger reduction of total EPS production than deletion of the whole levansucrase coding sequence. In both cases, metabolic activity of the mutant strains was increased, as indicated, for instance, by the maximum carbon dioxide transfer rate (CTR) reached during the fermentation process.

### SUMMARY OF THE INVENTION

In one aspect, provided herein is a Paenibacillus sp. strain comprising
a) no or a mutant PepR or b) a mutant PepH, or c) no or a mutant PepX, or
d) a mutant ManC, or e) a mutant SacB, or f) a combination of at least two of a), b), c), d) or e), wherein the missing PepR or the mutant PepR results in a decreased viscosity of a liquid culture of the Paenibacillus sp. strain in comparison to a liquid culture of a Paenibacillus sp. strain comprising a wildtype PepR and the mutant PepH results in a decreased viscosity of a liquid culture of the Paenibacillus sp. strain in comparison to a liquid culture of a Paenibacillus sp. strain comprising a wildtype PepH and the missing PepX or the mutant PepX results in a decreased viscosity of a liquid culture of the Paenibacillus sp. strain in comparison to a liquid culture of a Paenibacillus sp. strain comprising a wildtype PepX and the mutant ManC results in a decreased viscosity of a liquid culture of the Paenibacillus sp. strain in comparison to a liquid culture of a Paenibacillus sp. strain comprising a wildtype ManC or in comparison to a liquid culture of a Paenibacillus sp. strain comprising no ManC, and
the mutant SacB results in a decreased viscosity of a liquid culture of the Paenibacillus sp. strain in comparison to a liquid culture of a Paenibacillus sp. strain comprising no SacB.

In a further aspect, provided herein is an agricultural composition comprising a Paenibacillus sp. strain comprising a) no or a mutant PepR or b) no or a mutant PepH or c) no or a mutant PepX or d) no or a mutant ManC or e) no or a mutant SacB, or f) a combination of at least two of a), b), c), d), e) or f), wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB and
wherein the Paenibacillus sp. strain comprising the respective wildtype PepR, PepH, PepX, ManC or SacB and used for comparison is of the same Paenibacillus species than the strain comprising no PepR, PepH, PepX, ManC or SacB or a mutant thereof.

In a further aspect, it is provided herein, a plant propagation material comprising a Paenibacillus sp. strain comprising a) no or a mutant PepR or b) no or a mutant PepH or c) no or a mutant PepX or d) no or a mutant ManC or e) no or a mutant SacB, or f) a combination of at least two of a), b), c), d), e) or f), wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB and
wherein the Paenibacillus sp. strain comprising the respective wildtype PepR, PepH, PepX, ManC or SacB and used for comparison is of the same Paenibacillus species than the comprising no PepR, PepH, PepX, ManC or SacB or a mutant thereof.

In one aspect, provided herein is a method of suppressing or preventing fungal infection of a plant, wherein the fungi, their habitat or the materials or plants to be protected against fungal attack, or the soil or plant propagation material are treated with an effective amount of a Paeni-bacillus sp. strain comprising a) no or a mutant PepR or b) no or a mutant PepH or c) no or a mutant PepX or d) no or a mutant ManC or e) no or a mutant SacB, or f) a combination of at least two of a), b), c), d), e) or f), wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB, or
wherein the fungi, their habitat or the materials or plants to be protected against fungal attack, or the soil or plant propagation material are treated with an effective amount of an agricultural composition comprising a Paenibacillus sp. strain comprising a) no or a mutant PepR or b) no or a mutant PepH or c) no or a mutant PepX or d) no or a mutant ManC or e) no or a mutant SacB, or f) a combination of at least two of a), b), c), d), e) or f), wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB and
wherein the Paenibacillus sp. strain comprising the respective wildtype PepR, PepH, PepX, ManC or SacB and used for comparison is of the same Paenibacillus species than the comprising no PepR, PepH, PepX, ManC or SacB or a mutant thereof.

Provided is also a method of production of a valuable product via fermentation comprising:
1) culturing a Paenibacillus sp. strain comprising
   a) no or a mutant PepR or b) no or a mutant PepH or c) no or a mutant PepX or d) no or a mutant ManC or e) no or a mutant SacB, or f) a combination of at least two of a), b), c), d), e) or f), wherein
      the missing PepR or the mutant PepR results in a decreased viscosity of a liquid culture of the Paenibacillus sp. strain in comparison to a liquid culture of a Paenibacillus sp. strain comprising a wildtype PepR and
      the missing PepH or the mutant PepH results in a decreased viscosity of a liquid culture of the Paenibacillus sp. strain in comparison to a liquid culture of a Paenibacillus sp. strain comprising a wildtype PepH and
      the missing PepX or the mutant PepX results in a decreased viscosity of a liquid culture of the Paenibacillus sp. strain in comparison to a liquid culture of a Paenibacillus sp. strain comprising a wildtype PepX and
      the missing ManC or the mutant ManC results in a decreased viscosity of a liquid culture of the Paenibacillus sp. strain in comparison to a liquid culture of a Paenibacillus sp. strain comprising a wildtype ManC and
      the missing SacB or the mutant SacB results in a decreased viscosity of a liquid culture of the Paenibacillus sp. strain in comparison to a liquid culture of a Paenibacillus sp. strain comprising no SacB, wherein the Paenibacillus sp. strain comprising the respective wildtype PepR, PepH, PepX, ManC or SacB and used for comparison is of the same Paenibacillus species than the comprising no PepR, PepH, PepX, ManC or SacB or a mutant thereof.
      in a culture medium under conditions in which the valuable product is produced and
   2) harvesting the valuable product.

### BRIEF DESCRIPTION OF THE DRAWINGS

FIG. 1 depicts the development of viscosity over culture time of different mutants of Paenibacillus LU17007 and strains derived from Paenibacillus strain LU17007 by deleting the regions coding for PepR (here called exoT), SacB and a strain variant comprising a G323S mutant of SacB.

FIG. 2 depicts an alignment of the amino acid sequences of PepR of Paenibacillus strain LU17007, Paenibacillus ottowii, Paenibacillus polymyxa DSM365, Paenibacillus terrae, Paeni-bacillus kripbbensis and Paenibacillus sp. Aloe-11.

FIG. 3 depicts an alignment of the amino acid sequences of PepH of Paenibacillus strain LU17007, Paenibacillus ottowii, Paenibacillus polymyxa DSM365, Paenibacillus terrae, Paeni-bacillus kripbbensis and Paenibacillus sp. Aloe-11.

FIG. 4 depicts an alignment of the amino acid sequences of PepX of Paenibacillus strain LU17007, Paenibacillus ottowii, Paenibacillus polymyxa DSM365, Paenibacillus terrae, Paeni-bacillus kripbbensis and Paenibacillus sp. Aloe-11.

FIG. 5 depicts an alignment of the amino acid sequences of ManC of Paenibacillus strain LU17007, Paenibacillus ottowii, Paenibacillus polymyxa DSM365, Paenibacillus terrae, Paeni-bacillus kripbbensis and Paenibacillus sp. Aloe-11.

FIG. 6 depicts an alignment of the amino acid sequences of SacB of Paenibacillus strain LU17007, Paenibacillus ottowii, Paenibacillus polymyxa DSM365, Paenibacillus terrae, Paeni-bacillus kripbbensis and Paenibacillus sp. Aloe-11.

### DETAILED DESCRIPTION

Described herein are Paenibacillus strains comprising
a) no or a mutant PepR or
b) a mutant PepH, or
c) no or a mutant PepX, or
d) a mutant ManC, or
e) a mutant SacB, or
f) a combination of at least two of a), b), c), d) or e),
wherein
the missing PepR or the mutant PepR results in a decreased viscosity of a liquid culture of the Paenibacillus sp. strain in comparison to a liquid culture of a Paenibacillus sp. strain comprising a wildtype PepR and
the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the mutant ManC results in a decreased viscosity of a liquid culture of the Paenibacillus sp. strain in comparison to a liquid culture of a Paenibacillus sp. strain comprising a wildtype ManC or in comparison to a liquid culture of a Paenibacillus sp. strain comprising no ManC and
the mutant SacB results in a decreased viscosity of a liquid culture of the Paenibacillus sp. strain in comparison to a liquid culture of a Paenibacillus sp. strain comprising no SacB.

Preferably the Paenibacillus sp. strain comprising the respective wildtype PepR, PepH, PepX, ManC or SacB and used for comparison is of the same Paenibacillus species than the comprising no PepR, PepH, PepX, ManC or SacB or a mutant thereof.

Even more preferred the Paenibacillus sp. strain used for comparison is the parent strain of the Paenibacillus lacking a PepR, PepH, PepX, ManC or SacB or comprising the respective mutant.

Preferably, a strain comprising no PepR, PepH, PepX, ManC or SacB has a deletion of the respective gene sequence or a mutation leading to a premature stop codon.

As used herein, the term Paenibacillus strain is identical to the term Paenibacillus sp. strain and means a bacterial strain form the genus Paenibacillus. The genus Paenibacillus includes all species Paenibacillus spp.

These Paenibacillus strains disclosed herein show a decreased viscosity when grown in a liquid culture when compared to a Paenibacillus strain comprising a wildtype PepR, PepH or SacB, respectively.

A Paenibacillus strain comprising no or a mutant PepR is usually compared to a Paenibacillus strain comprising a wildtype PepR. Preferably the Paenibacillus strain comprising no or a mutant PepR is compared to the Paenibacillus strain from which it was produced via mutation or transgenic or gene editing methods, i.e. the parent strain.

Accordingly, the parent strain has an identical genome sequence to the respective Paenibacillus strain, except for the presence of the respective mutation.

The same principle applies to Paenibacillus strains comprising a mutant PepH or a mutant SacB.

The viscosity is preferably measured using the same methods as described in Examples 2 and 3 herein, wherein either glucose or saccharose, preferably glucose, is used as C-source. Preferably the liquid medium comprises glucose as described in Example 2. A strain is considered to produce less viscosity when grown in liquid culture, when the viscosity [mPa·s] measured at 100/s is lower than the viscosity [mPa·s] measured at 100/s of the comparison strain after a culturing time of at least 12, at least 24 hours, or at least 40 hours upon the initiation of microbial growth, preferably after a culturing time of at least 24 hours. Preferably, a strain is considered to produce less viscosity, when the sum of the viscosities measured every hour between 8 hours of culture and 40 hours of culture is less than the sum of the viscosities measured every hour between 8 hours of culture and 40 hours of culture of the strain used for comparison.

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

The term “strain” is intended to refer to microbial cells which can be considered genetically homogenous when the natural frequency of mutation during growth of the cells is taken into account. Strains are often isolated via culture of microbial colonies from one cell.

Microbial cultures grown from one strain are considered to be biological pure cultures.

As used herein, the term “biologically pure culture” refers to a culture of a strain, which is essentially free of microorganisms of other species or strains. In a preferred embodiment a “biologically pure culture” comprises less than 1%, more preferred less than 0.1%, even more preferred less than 0.01%, of cells of other species.

Strains can also intentionally be mixed with other strains to create co-cultures. Such co-cultures are considered to comprise one or more biologically pure culture of the individual strains at the same time.

The term mutant, when used in respect to a protein, refers to the situation in which the genomic region coding for such protein comprises at least one nucleotide which results in an altered amino acid sequence of the encoded protein in comparison to the wildtype amino acid sequence of such protein.

The term mutant, when used in respect to the gene of a protein, refers to the situation in which the polynucleotide sequence of the promoter or encoded RNA sequence for such protein differs from the wildtype sequence and results in an altered, preferably lower, expression level of the encoded protein.

In one embodiment of the invention, the Paenibacillus strains comprise a mutant gene, which results in a lower expression level of PepR, PepH, PepX, ManC and/or SacB in such Paeni-bacillus strain in comparison to the wildtype of this Paenibacillus strain.

The Paenibacillus strains of the invention can be cultivated continuously or discontinuously in the batch process or in the fed batch or repeated fed batch process. A review of known methods of cultivation will be found in the textbook by Chmiel (Bioprozesstechnik 1. Einfuhrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)). The medium that is to be used for cultivation of the Paenibacillus strain must satisfy the requirements of the particular strain in an appropriate manner. Descriptions of culture media for various microorganisms are given in the handbook “Manual of Methods for General Bacteriology” of the American Society for Bacteriology (Washington D. C., USA, 1981). Further suitable media are disclosed in the art, e.g. in the documents cited herein.

Preferably the Paenibacillus strains belong to the Paenibacillus species: Paenibacillus polymyxa, Paenibacillus jamilae, Paenibacillus ottowii, Paenibacillus terrae, or Paenibacillus kribensis.

Preferred strains suitable to construct strains of the invention are P. polymyxa strain DSM365, P. polymyxa strain PKB1, P. polymyxa strain JB05-01-1, P. polymyxa strain AC-1, P. polymyxa strain HY96-2, Paenibacillus sp. Aloe-11, Paenibacillus sp. strains NRRL B-50972, NRRL B67129, NRRL B-67304, NRRL B-67306 and NRRL B-67615, NRRL B-50374, NRRL B-67721, NRRL B-67723, NRRL B-67724, P. polymyxa strain VMC10/96, Paenibacillus sp. strain 10.6D, Paenibacillus sp. strain 9.4E, Paenibacillus sp. strains Lu16774, Lu17007 and Lu17015, P. polymyxa strain M1, P. polymyxa strain SC2, P. polymyxa strain Sb3-1 and P. polymyxa strain E681. In particular preferred strains are Lu17007 and DSM365.

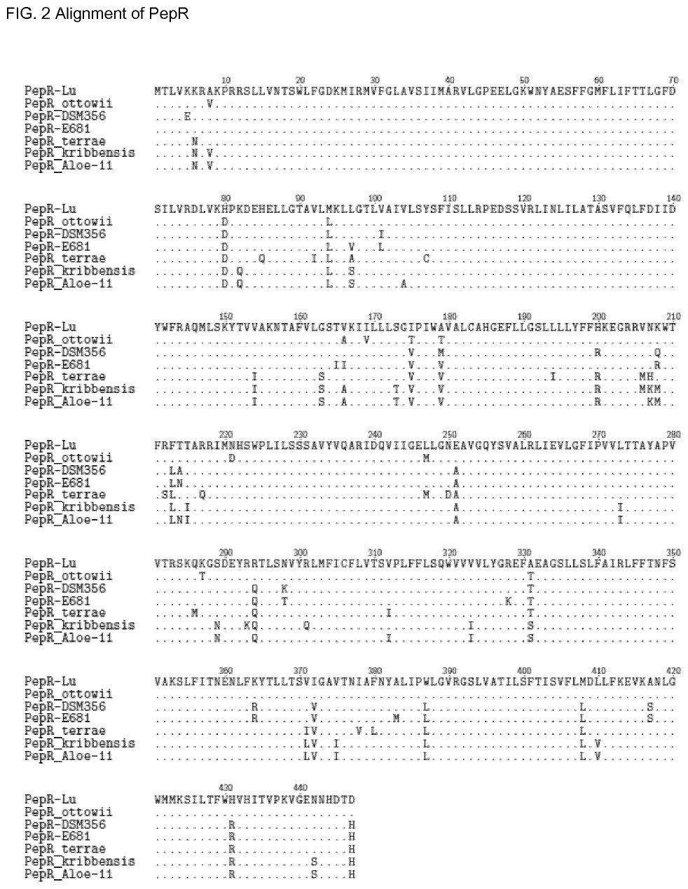

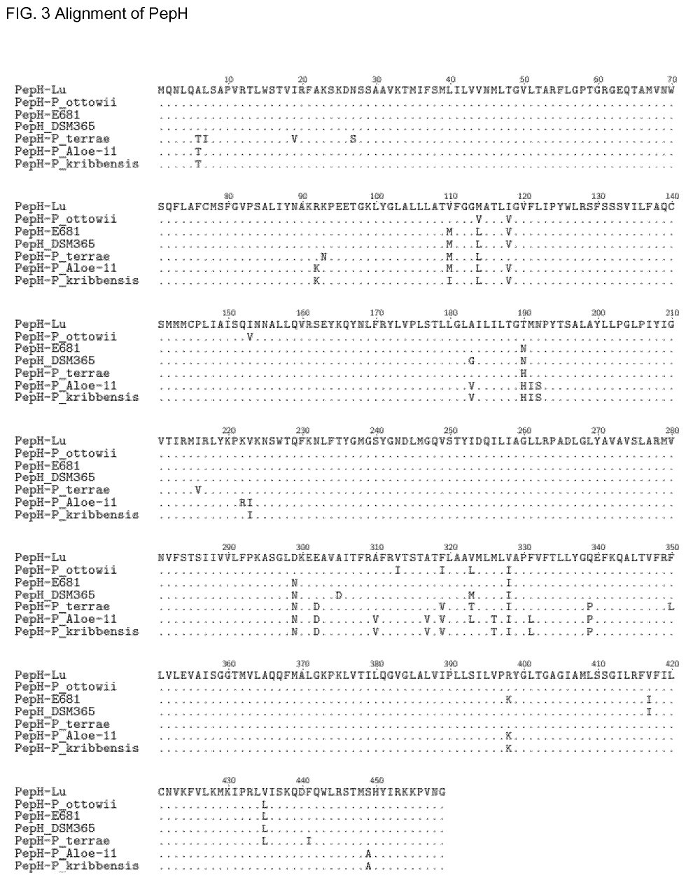

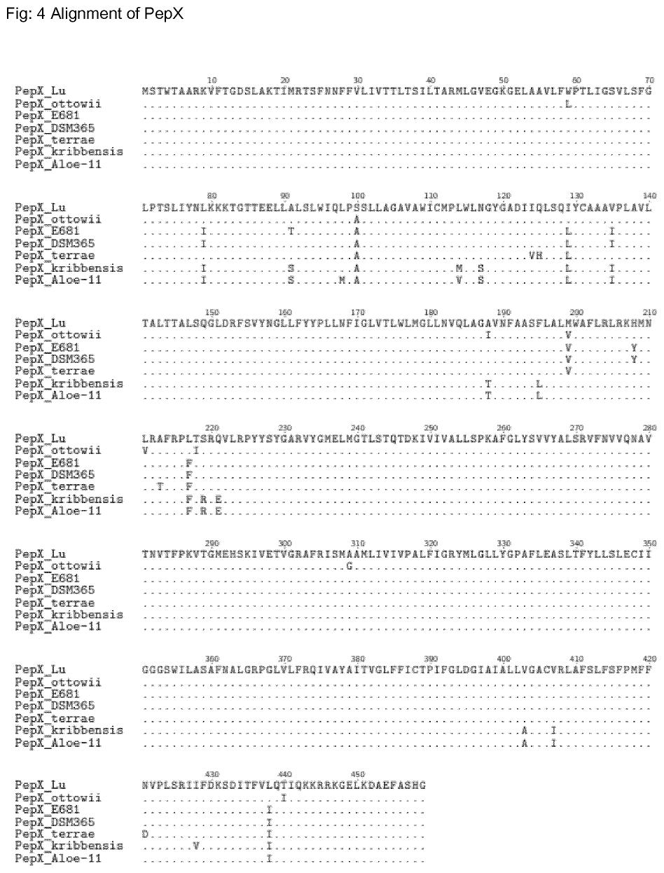

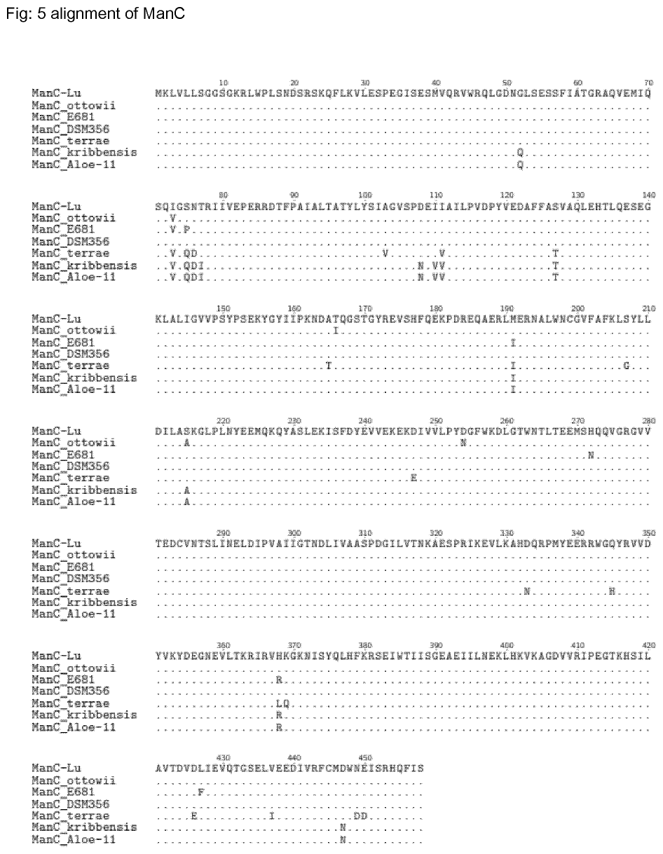

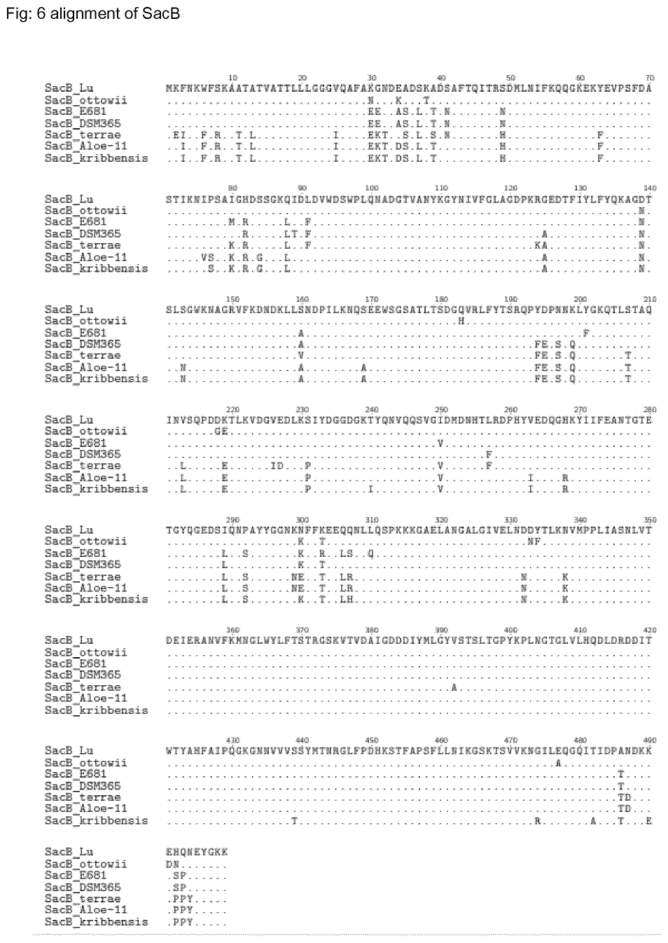

Paenibacillus strains comprising no or a mutant flippase PepR or a mutant flippase PepH, no or a mutant flippase PepX, no or a mutant mannose-1-phosphate guanylyl transferase ManC, or a mutant levansucrase SacB are preferably strains in which the wildtype PepR has an amino acid sequence which is at least 80%, 90%, 91%, 92%, 93%, 94%, 95%, 96%, 97%, 98%, 99% or 100% identical to Seq ID No. 1 and the mutant PepR comprises an amino acid, which differs from the amino acid at the same place of the amino acid which is 100% conserved in the alignment shown in FIG. 2, or
wherein the wildtype PepH has an amino acid sequence which is at least 80%, 90%, 91%, 92%, 93%, 94%, 95%, 96%, 97%, 98%, 99% or 100% identical to Seq ID No. 8, and the mutant PepH comprises an amino acid, which differs from the amino acid at the same place of the amino acid which is 100% conserved in the alignment shown in FIG. 3, or
wherein the wildtype PepX has an amino acid sequence which is at least 80%, 90%, 91%, 92%, 93%, 94%, 95%, 96%, 97%, 98%, 99% or 100% identical to Seq ID No. 15, and the mutant PepH comprises an amino acid, which differs from the amino acid at the same place of the amino acid which is 100% conserved in the alignment shown in FIG. 4, or
wherein the wildtype ManC has an amino acid sequence which is at least 80%, 90%, 91%, 92%, 93%, 94%, 95%, 96%, 97%, 98%, 99% or 100% identical to Seq ID No. 22, and the mutant ManC comprises an amino acid, which differs from the amino acid at the same place of the amino acid which is 100% conserved in the alignment shown in FIG. 5, or
wherein the wildtype SacB has an amino acid sequence which is at least 80%, 90%, 91%, 92%, 93%, 94%, 95%, 96%, 97%, 98%, 99% or 100% identical to Seq ID No. 29 and the mutant SacB comprises an amino acid, which differs from the amino acid at the same place of the amino acid which is 100% conserved in the alignment shown in FIG. 6.

Paenibacillus strains comprising no or a mutant flippase PepR or a mutant flippase PepH, no or a mutant flippase PepX, no or a mutant mannose-1-phosphate guanylyl transferase ManC, or a mutant levansucrase SacB are preferably strains in which the wildtype PepR has an amino acid sequence which is at least 80%, 90%, 91%, 92%, 93%, 94%, 95%, 96%, 97%, 98%, 99% or 100% identical to Seq ID No. 1 and the mutant PepR comprises an amino acid sequence, which is not 100% identical with any one of Seq. ID No. 1 to 7 or comprises a premature stop codon, or
wherein the wildtype PepH has an amino acid sequence which is at least 80%, 90%, 91%, 92%, 93%, 94%, 95%, 96%, 97%, 98%, 99% or 100% identical to Seq ID No. 8, and the mutant PepR comprises an amino acid sequence, which is not 100% identical with any one of Seq. ID No. 8 to 14 or comprises a premature stop codon, or
wherein the wildtype PepX has an amino acid sequence which is at least 80%, 90%, 91%, 92%, 93%, 94%, 95%, 96%, 97%, 98%, 99% or 100% identical to Seq ID No. 15, and the mutant PepX comprises an amino acid sequence, which is not 100% identical with any one of Seq. ID No. 15 to 21 or comprises a premature stop codon, or
wherein the wildtype ManC has an amino acid sequence which is at least 80%, 90%, 91%, 92%, 93%, 94%, 95%, 96%, 97%, 98%, 99% or 100% identical to Seq ID No. 22, and the mutant ManC comprises an amino acid sequence, which is not 100% identical with any one of Seq. ID No. 22 to 28 or comprises a premature stop codon, or
wherein the wildtype SacB has an amino acid sequence which is at least 80%, 90%, 91%, 92%, 93%, 94%, 95%, 96%, 97%, 98%, 99% or 100% identical to Seq ID No. 29 and the mutant SacB comprises an amino acid sequence, which is not 100% identical with any one of Seq. ID No. 29 to 35 or comprises a premature stop codon.

In one embodiment the Paenibacillus strain comprises a mutant PepR comprising a W224* or a S393F mutation.

In one embodiment the Paenibacillus strain comprises a mutant PepH comprising a E96K mutation or a E163K mutation or a E96K and a E163K mutation.

In one embodiment the Paenibacillus strain comprises a mutant PepH and no or a mutant PepR.

In one embodiment the Paenibacillus strain comprises a mutant PepH comprising a E96K mutation or a E163K mutation or a E96K and a E163K mutation and a mutant PepR comprising a W224* or S393F mutation.

In one embodiment the Paenibacillus strain comprises a mutant ManC comprising a P90S, E340K, or G433D mutation

In one embodiment the Paenibacillus strain comprises a mutant SacB comprising a G323S mutation.

The Paenibacillus strains, can be grown in culture broth and under culture conditions which are well known to the skilled person and disclosed for example in WO16020371 of which pages 20, line 33 to page 22, line 29 are hereby included by reference.

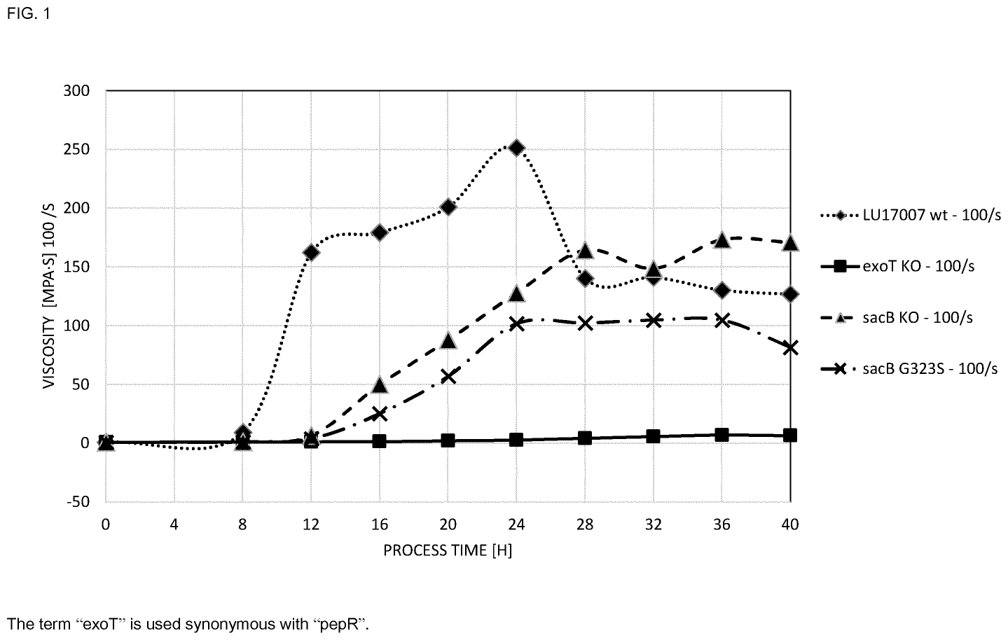

Paenibacilli can be used as biological pesticides to suppress or prevent pathogen infection of plants. Paenibacilli strains suitable to suppress or prevent pathogen infection are disclosed for example in WO2016/154297, WO2019/221988, WO2020/181053, WO2019/155253, WO2018/195603. Many Paenibacillus strains which are capable to suppress or prevent pathogen infection of plants produce Fusarididins. Accordingly, the strains disclosed herein are preferably created from Fusaricidin producing strains. Fusaricidins are a group of antibiotics isolated from Paenibacillus spp. from the class of cyclic lipodepsipeptides which often share the following structural features: a macrocyclic ring consisting of 6 amino acid residues, three of which are L-Thr, D-allo-Thr and D-Ala, as well as the 15-guanidino-3-hydroxypentadecanoic acid tail attached to the N-terminal L-Thr residue by an amide bond (ChemMedChem 7, 871-882, 2012; J. Microbiol. Meth. 85, 175-182, 2011, Table 1 herein). These compounds are cyclized by a lactone bridge between the N-terminal L-Thr hydroxyl group and the C-terminal D-Ala carbonyl group. The position of the amino acid residues within the depsipeptide cycle are usually numbered starting with the abovementioned L-Thr which itself also carries the GHPD chain and ending with the C-terminal D-Ala. Non-limiting examples of fusaricidins isolated from Paenibacillus are designated LI-F03, LI-F04, LI-F05, LI-F07 and LI-F08 (J. Antibiotics 40(11), 1506-1514, 1987; Heterocycles 53(7), 1533-1549, 2000; Peptides 32, 1917-1923, 2011) and fusaricidins A (also called LI-F04a), B (also called LI-F04b), C (also called LI-F03a) and D (also called LIF03b) (J. Antibiotics 49(2), 129-135, 1996; J. Antibiotics 50(3), 220-228, 1997). The amino acid chain of a fusaricidin is not ribosomally generated but is generated by a non-ribosomal peptide synthetase. Structural formulae of known fusaricidins are shown in Table 1 (Biotechnol Lett. 34, 1327-1334, 2012; FIG. 1 therein). The compounds designated as LI-F03a, LI-F03b up to LI-F08a and LI-F08b and the fusaricidins of formulae I and 1.1 as described herein are also referred to as fusaricidins LI-F03a, LI-F03b up to LI-F08a and LI-F08b due to their structure within the fusaricidin family (see e.g. Table 1).

Among isolated fusaricidin antibiotics, fusaricidin A has shown the most promising antimicrobial activity against a variety of clinically relevant fungi and gram-positive bacteria such a Staphylococcus aureus (MIC value range: 0.78-3.12 μg/ml) (ChemMedChem 7, 871-882, 2012). The synthesis of fusaricidin analogues that contain 12-guanidino-dodecanoic acid (12-GDA) or 12-amino-dodecanoic acid (12-ADA) instead of naturally occurring GHPD has been established but the replacement of GHPD by 12-ADA resulted in complete loss of the antimicrobial activity while the replacement of GHPD by 12-GDA retained antimicrobial activity (Tetrahedron Lett. 47, 8587-8590, 2006; ChemMedChem 7, 871-882, 2012).

TABLE 1
Structures of the fusaricidin family.

| Fusaricidin | X² | X³ | X⁵ |
|---|---|---|---|
| A (LI-F04a) | D-Val | L-Val | D-Asn |
| B (LI-F04b) | D-Val | L-Val | D-Gln |
| C (LI-F03a) | D-Val | L-Tyr | D-Asn |
| D (LI-F03b) | D-Val | L-Tyr | D-Gln |
| LI-F05a | D-Val | L-lle | D-Asn |
| LI-F05b | D-Val | L-lle | D-Gln |
| LI-F06a | D-allo-lle | L-Val | D-Asn |
| LI-F06b | D-allo-lle | L-Val | D-Gln |
| LI-F07a | D-Val | L-Phe | D-Asn |
| LI-F07b | D-Val | L-Phe | D-Gln |
| LI-F08a | D-Ile | L-allo-lle | D-Asn |
| LI-F08b | D-Ile | L-allo-lle | D-Gln |

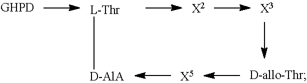

wherein an arrow defines a single (amide) bond either between the carbonyl moiety of GHPD and the amino group of L-Thr (L-threonine) or between the carbonyl group of one amino acid and the amino group of a neighboring amino acid, wherein the tip of the arrow indicates the attachment to the amino group of said amino acid L-Thr or of said neighboring amino acid; and
wherein the single line without an arrow head defines a single (ester) bond between the carbonyl group of D-Ala (D-alanine) and the hydroxyl group of L-Thr; and wherein GHPD is 15-guanidino-3-hydroxypentadecanoic acid.

Fusaricidins A, B, C and D are also reported to inhibit plant pathogenic fungi such as Fusarium oxysporum, Aspergillus niger, Aspergillus oryzae, and Penicillum thomii (J. Antibiotics 49(2), 129-135, 1996; J. Antibiotics 50(3), 220-228, 1997). Fusaricidins such as Li-F05, LI-F07 and LI-F08 have been found to have certain antifungal activity against various plant pathogenic fungi such as Fusarium moniliforme, F. oxysporum, F. roseum, Giberella fujkuroi, Helminthosporium sesamum and Penicillium expansum (J. Antibiotics 40(11), 1506-1514, 1987). Fusaricidins also have antibacterial activity to Gram-positive bacteria including Staphylococcus aureus (J. Antibiotics 49, 129-135, 1996; J. Antibiotics 50, 220-228, 1997). In addition, fusaricidins have antifungal activity against Leptosphaeria maculans which causes black root rot of canola (Can. J. Microbiol. 48, 159-169, 2002). Moreover, fusaricidins A and B and two related compounds thereof, wherein D-allo-Thr is bound via its hydroxyl group to an additional alanine using an ester bridge, produced by certain Paenibacillus strains were found to induce resistance reactions in cultured parsley cells and to inhibit growth of Fusarium oxysporum (WO 2006/016558; EP 1 788 074 A1).

WO 2007/086645 describes a fusaricidin synthetase enzyme and its encoding gene as isolated from Paenibacillus polymyxa strain E681. The fusaricidin synthetase and its homologs in other Paenibacilli species are involved in the synthesis of fusaricidins A, B, C, D, LI-F03, LI-F04, LI-F05, LI-F07 and LI-F08.

Accordingly, the invention comprises also agricultural compositions comprising a Paenibacillus sp. strain comprising
a) no or a mutant PepR or
b) no or a mutant PepH or
c) no or a mutant PepX or
d) no or a mutant ManC or
e) no or a mutant SacB, or
f) a combination of at least two of a), b), c), d), e) or f)
   wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB, and
wherein the Paenibacillus sp. strain comprising the respective wildtype PepR, PepH, PepX, ManC or SacB and used for comparison is of the same Paenibacillus species than the strain comprising no PepR, PepH, PepX, ManC or SacB or a mutant thereof.

Preferably the agrochemical composition comprises a mutant PepR comprising a W224* or a S393F mutation.

Preferably the agrochemical composition comprises a mutant PepH comprising a E96K mutation or a E163K mutation or a E96K and a E163K mutation.

Preferably the agrochemical composition comprises a mutant a mutant PepH and no or a mutant PepR.

Preferably the agrochemical composition comprises a mutant PepH comprising a E96K mutation or a E163K mutation or a E96K and a E163K mutation and a mutant PepR comprising a W224* or S393F mutation

Preferably the agrochemical composition comprises a mutant ManC comprising a P90S, E340K, or G433D mutation.

Preferably the agrochemical composition comprises a mutant SacB comprising a G323S mutation.

The agricultural compositions are preferably customary types of agrochemical compositions, e.g. solutions, emulsions, suspensions, dusts, powders, pastes, granules, pressings, capsules, and mixtures thereof. Examples for composition types (see also “Catalogue of pesticide formulation types and international coding system”, Technical Monograph No. 2, 6^{th} Ed. May 2008, CropLife International) are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials, such as seeds (e.g. GF). The compositions are prepared in a known manner, such as described by Mollet and Grubemann, Formulation technology, Wiley VCH, Weinheim, 2001; or by Knowles, New developments in crop protection product formulation, Agrow Reports DS243, T&F Informa, London, 2005. The invention also relates to agrochemical compositions comprising an Paenibacillus strain of the invention and an auxiliary.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers, and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, and alkylated naphthalenes; alcohols, e.g. ethanol, propanol, butanol, benzyl alcohol, cyclohexanol, glycols; DMSO; ketones, e.g. cyclohexanone; esters, e.g. lactates, carbonates, fatty acid esters, gamma-butyrolactone; fatty acids; phosphonates; amines; amides, e.g. N-methyl pyrrolidone, fatty acid dimethyl amides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and of alkylnaphthalenes, sulfosuccinates, or sulfosuccinamates. Examples of sulfates are sulfates of fatty acids, of oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose and glucose esters, or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide, and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethyl cellulose), inorganic clays (organically modified or unmodified), polycarboxylates, and silicates.

Suitable bactericides are bronopol and isothiazolinone derivatives, such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

The agrochemical compositions generally comprise between 0.01 and 95%, preferably between 0.1 and 90%, more preferably between 1 and 70%, and in particular between 10 and 60%, by weight of cells or spores of the Paenibacillus strain.

The amount of these cells or spores is preferably between 5% w/w and 50% w/w, 10% w/w and 50% w/w, 15% w/w and 50% w/w, 30% w/w and 50% w/w, or 40% w/w and 50% w/w, or between 5% w/w and 40% w/w, 10% w/w and 40% w/w, 15% w/w and 40% w/w, 30% w/w and 40% w/w, or between 10% w/w and 40% w/w, 15% w/w and 40% w/w, 30% w/w and 40% w/w of the agrochemical composition.

The cells or spores of the Paenibacillus strains are usually present in the form of solid particles having an average particle size of 1 to 150 μm, or in an increased order of preference of 1 to 100 μm, 1 to 75 μm, 1 to 50 μm, 1 to 25 μm, 1 to 10 μm, or 1 to 8 μm (determined according to light scattering method in liquid dispersion according to CIPAC method 187).

The density number of spores per ml can be determined by identifying the number of colonyforming units (CFU) on agar medium e.g. potato dextrose agar after incubation for several days at temperatures of about 20 to about 35° C. The amount of CFU/g of biomass used to prepare agrochemical compositions of the invention are usually between 1×10⁸ CFU/g to 1×10¹¹ CFU/g, or 1×10⁸ CFU/g to 1×10¹⁰ CFU/g, or 5×10⁸ to 5×10¹⁰ CFU/g, preferably between 1×10⁹ CFU/g to 1×10¹⁰ CFU/g. The CFU/g of biomass will influence the amount of biomass which is used to prepare the formulations of the invention. Biomass having a comparatively high amount of CFU/g can be used to prepare formulations having a comparatively low amount of biomass. The amount of biomass used for preparing the formulations of the invention is usually selected to fit the amount of CFU per hectare, which should be applied for the respective purpose.

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying the mixtures or agrochemical compositions comprising the mixtures, respectively, onto young plants and propagation material like seedlings, rooted/unrooted cuttings, plants derived from cell-culture, include dressing, coating, pelleting, dusting, soaking, as well as in-furrow application methods. Preferably, the mixtures and agrochemical compositions thereof, respectively, are applied on to seeds by a method such that germination is not induced, e.g. by seed dressing, pelleting, coating, and dusting.

Various types of oils, wetters, adjuvants, fertilizers, or micronutrients, and further pesticides (e.g. fungicides, growth regulators, herbicides, insecticides, safeners) may be added to the mixtures or the agrochemical compositions thereof as premix, or, not until immediately prior to use (tank mix). These agents can be admixed with the mixturs or the agrochemical compositions according to the invention in a weight ratio of 1:100 to 100:1, preferably 1:10 to 10:1.

The Paenibacillus strains and the agrochemical composition comprising these strains ae usually applied in fungicidally active amounts. The term “fungicidally effective amount” denotes an amount of the composition or of the mixtures, which is sufficient for controlling harmful fungi plants and which does not result in a substantial damage to the treated plants, young plants like seedlings, rooted/unrooted cuttings, plants derived from cell-culture or plant propagation materials, such as seeds. Such an amount can vary in a broad range and is dependent on various factors, such as the fungal species to be controlled, the treated plant species, the climatic conditions and the specific mixture used.

When the agrochemical compositions are used to be applied as foliar treatment or to the soil, preferably as foliar treatment. The application rates in foliar treatments are usually between 50 g/ha and 2000 g/ha, 100 g/ha and 2000 g/ha, 150 g/ha and 2000 g/ha, 600 g/ha and 2000 g/ha or 800 g/ha and 2000 g/ha or
between 50 g/ha and 1000 g/ha, 100 g/ha and 1000 g/ha, 150 g/ha and 1000 g/ha, 600 g/ha and 1000 g/ha or 800 g/ha and 1000 g/ha,
or between 50 g/ha and 800 g/ha, 100 g/ha and 800 g/ha, 150 g/ha and 800 g/ha, 600 g/ha and 800 g/ha or
between 150 g/ha and 1000 g/ha, 300 g/ha and 1000 g/ha, 600 g/ha and 1000 g/ha of fusaricidin comprising cells or spores in a volume
between 1000 L/ha and 100 L/ha, 600 L/ha and 100 L/ha, 400 L/ha and 100 L/ha, 200 L/ha and 100 L/ha or
between 1000 L/ha and 600 L/ha, 1000 L/ha and 400 L/ha or 1000 L/ha and 200 L/ha, or between 600 L/ha and 200 L/ha, 600 L/ha and 400 L/ha of a water based spraying liquid.

When the agrochemical compositions are employed in seed treatment, for example as seed coating, the application rates with respect to plant propagation material usually range from about 1×10¹ to 1×10¹² (or more) CFU/seed, preferably from about 1×10³ to about 1×10¹⁰ CFU/seed, and even more preferably from about 1×10³ to about 1×10⁶ CFU/seed. Alternatively, the application rates with respect to plant propagation material preferably range from about 1×10⁷ to 1×10¹⁶ (or more) CFU per 100 kg of seed, preferably from 1×10⁹ to about 1×10¹⁵ CFU per 100 kg of seed, even more preferably from 1×10¹¹ to about 1×10¹⁵ CFU per 100 kg of seed.

The Paenibacillus strains and the agrochemical compositions comprising these strains, respectively, are suitable as fungicides effective against a broad spectrum of phytopathogenic fungi, including soil-borne fungi, in particular from the classes of Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). They can be used in crop protection as foliar fungicides, fungicides for seed dressing, and soil fungicides.

The mixtures and the agrochemical compositions thereof are preferably useful in the control of phytopathogenic fungi on various cultivated plants, such as cereals, e.g. wheat, rye, barley, triticale, oats, or rice; beet, e.g. sugar beet or fodder beet; fruits, e.g. pomes (apples, pears, etc.), stone fruits (e.g. plums, peaches, almonds, cherries), or soft fruits, also called berries (strawberries, raspberries, blackberries, gooseberries, etc.); leguminous plants, e.g. lentils, peas, alfalfa, or soybeans; oil plants, e.g. oilseed rape, mustard, olives, sunflowers, coconut, cocoa beans, castor oil plants, oil palms, ground nuts, or soybeans; cucurbits, e.g. squashes, cucumber, or melons; fiber plants, e.g. cotton, flax, hemp, or jute; citrus fruits, e.g. oranges, lemons, grapefruits, or mandarins; vegetables, e.g. spinach, lettuce, asparagus, cabbages, carrots, onions, tomatoes, potatoes, cucurbits, or paprika; lauraceous plants, e.g. avocados, cinnamon, or camphor; energy and raw material plants, e.g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e.g. flowers, shrubs, broad-leaved trees, or evergreens (conifers, eucalypts, etc.); on the plant propagation material, such as seeds; and on the crop material of these plants.

More preferably, the mixtures and the agrochemical compositions thereof, respectively, are used for controlling fungi on field crops, such as potatoes, sugar beets, tobacco, wheat, rye, barley, oats, rice, corn, cotton, soybeans, oilseed rape, legumes, sunflowers, coffee or sugar cane; fruits; vines; grapes for wine making or fruit grapes, ornamentals; or vegetables, such as cucumbers, tomatoes, pepper, beans or squashes.

The term “plant propagation material” is to be understood to denote all the generative parts of the plant, such as seeds; and vegetative plant materials, such as cuttings and tubers (e.g. potatoes), which can be used for the multiplication of the plant. This includes seeds, roots, fruits, tubers, bulbs, rhizomes, shoots, sprouts and other parts of plants; including seedlings and young plants to be transplanted after germination or after emergence from soil.

Accordingly, one embodiment of the invention is plant propagation material comprising the mixtures or comprising a coating of an agrochemical composition comprising the mixtures. Preferably the plant propagation material are young plants, like seedlings, rooted/unrooted cuttings, plants derived from cell-culture. Even more preferred the plant propagation material is from fruit or vegetable plant species, including grapes.

According to the invention all of the above cultivated plants are understood to comprise all species, subspecies, variants and/or hybrids which belong to the respective cultivated plants. For example, corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var. ceratina), amylomaize (high amylose Zea mays varieties), pod corn or wild maize (Zea mays var. tunicata) and striped maize (Zea mays var. japonica). The skilled person knows similar variants of other plant species, like, sweet peper, determinate or indeterminate soybean, and others.

The mixtures and the agrochemical compositions thereof, respectively, are particularly suitable for controlling the following causal agents of plant diseases: Albugo spp. (white rust) on ornamentals, vegetables (e.g. A. candida) and sunflowers (e.g. A. tragopogonis); Alternaria spp. (Alternaria leaf spot) on vegetables (e.g. A. dauci or A. porri), oilseed rape (A. brassicicola or brassicae), sugar beets (A. tenuis), fruits (e.g. A. grandis), rice, soybeans, potatoes and tomatoes (e.g. A. solani, A. grandis or A. alternata), tomatoes (e.g. A. solani or A. alternata) and wheat (e.g. A. triticina); Aphanomyces spp. on sugar beets and vegetables; Ascochyta spp. on cereals and vegetables, e.g. A. tritici (anthracnose) on wheat and A. hordei on barley; Aureobasidium zeae (syn. Kapatiella zeae) on corn; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages); B. squamosa or B. allii on onion family), oilseed rape, ornamentals (e.g. B. eliptica), vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladobotryum (syn. Dactylium) spp. (e.g. C. mycophilum (formerly Dactylium dendroides, teleomorph: Nectria albertinii, Nectria rosella syn. Hypomyces rosellus) on mushrooms; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: He/minthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum), soybeans (e.g. C. truncatum or C. gloeosporioides), vegetables (e.g. C. lagenarium or C. capsici), fruits (e.g. C. acutatum), coffee (e.g. C. coffeanum or C. kahawae) and C. gloeosporioides on various crops; Corticium spp., e.g. C. sasakii (sheath blight) on rice; Corynespora cassiicola (leaf spots) on soybeans, cotton and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. on pome fruits (E. pyn), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, oilseed rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals, potatoes and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (syn. Monilia spp.: bloom and twig blight, brown rot) on stone fruits and other rosaceous plants; Mycosphaerella spp. on cereals, bananas, soft fruits and ground nuts, such as e.g. M. graminicola (anamorph: Zymoseptoria tritici formerly Septoria tritici: Septoria blotch) on wheat or M. fijiensis (syn. Pseudocercospora fijiensis: black Sigatoka disease) and M. musicola on bananas, M. arachidicola (syn. M. arachidis or Cercospora arachidis), M. berkeleyi on peanuts, M. pisi on peas and M. brassiciola on brassicas; Peronospora spp. (downy mildew) on cabbage (e.g. P. brassicae), oilseed rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (syn. Leptosphaeria biglobosa and L. maculans: root and stem rot) on oilseed rape and cabbage, P. betae (root rot, leaf spot and damping-off) on sugar beets and P. zeae-maydis (syn. Phyllostica zeae) on corn; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, oilseed rape, radish and other plants; Plasmopara spp., e.g. P. viticola (grapevine downy mildew) on vines and P. halstediion sunflowers; Podosphaera spp. (powdery mildew) on rosaceous plants, hop, pome and soft fruits (e.g. P. leucotricha on apples) and curcurbits (P. xanthii); Polymyxa spp., e.g. on cereals, such as barley and wheat (P. graminis) and sugar beets (P. betae) and thereby transmitted viral diseases; Pseudocercosporella herpotrichoides (syn. Oculimacula yallundae, O. acuformis: eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or ‘rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenopeziza spp., e.g. P. brassicae on oilseed rape; Pyrenophora (anamorph: Drechslera) tritici-repentis (tan spot) on wheat or P. teres (net blotch) on barley; Pyricularia spp., e.g. P. oryzae (teleomorph: Magnaporthe grisea: rice blast) on rice and P. grisea on turf and cereals; Pythium spp. (damping-off) on turf, rice, corn, wheat, cotton, oilseed rape, sunflowers, soybeans, sugar beets, vegetables and various other plants (e.g. P. ultimum or P. aphanidermatum) and P. oligandrum on mushrooms; Ramularia spp., e.g. R. collo-cygni (Ramularia leaf spots, Physiological leaf spots) on barley, R. areola (teleomorph: Mycosphaerella areola) on cotton and R. beticola on sugar beets; Rhizoctonia spp. on cotton, rice, potatoes, turf, corn, oilseed rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis and R. commune (scald) on barley, rye and triticale; Sarocladium oryzae and S. attenuatum (sheath rot) on rice; Sclerotinia spp. (stem rot or white mold) on vegetables (S. minor and S. sclerotiorum) and field crops, such as oilseed rape, sunflowers (e.g. S. sclerotiorum) and soybeans, S. rolfsii (syn. Athelia rolfsii) on soybeans, peanut, vegetables, corn, cereals and ornamentals; Septoria spp. on various plants, e.g. S. glycines (brown spot) on soybeans, S. tritici (syn. Zymoseptoria tritici, Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setosphaeria spp. (leaf blight) on corn (e.g. S. turcicum, syn. Helminthosporium turcicum) and turf; Sphacelotheca spp. (smut) on corn, (e.g. S. reiliana, syn. Ustilago reiliana: head smut), sorghum und sugar cane; Sphaerotheca fuliginea (syn. Podosphaera xanthii: powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum, syn. Septoria nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Trichoderma harzianum on mushrooms; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli), sugar beets (e.g. U. betae or U. beticola) and on pulses (e.g. U. vignae, U. pisi, U. viciae-fabae and U. fabae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. longisporum on oilseed rape, V. dahliae on strawberries, oilseed rape, potatoes and tomatoes, and V. fungicola on mushrooms; Zymoseptoria tritici on cereals.

The mixtures and the agrochemical compositions thereof, respectively, are particularly suitable for controlling the following phytophathogenic fungi genera; Alternaria, Botrytis, Venturia, Leptosphaeria, Fusarium, Rhizoctonia, Phytophthora, Pythium, Colletotrichum, Pyricularia, Sclerotinia, Zymoseptoria.

The mixtures and the agrochemical compositions thereof, respectively, are particularly suitable for controlling the following phytophathogenic fungi, Alternaria solanum, Alternaria alternata, Alternaria brassicae, Alternaria brassicicola, Alternaria citri, Alternaria mali, Botrytis cinerea, Botrytis allii, Botrytis fabae, Botrytis squamosa, Venturia inaequalis, Venturia effusa, Venturia carpophila, Venturia pyrina, Leptosphaeria maculans, Leptosphaeria nodorum Fusarium oxysporum, Fusarium graminearum, Fusarium verticillioides, Rhizoctonia solani, Phytophthora infestans, Phytophthora capsici, Phytophthora fragariae, Phytophthora nicotianae, Phytophthora sojae, Pythium ultimum, Pythium acanthicum, Pythium deliense, Pythium graminicola, Pythium heterothallicum, Pythium hypogynum, Pythium middletonii Colletotrichum orbiculare, Colletotrichum capsici, Colletotrichum coccodes, Colletotrichum fragariae, Colletotrichum lindemuthianum, Pyricularia oryzae, Sclerotinia sclerotiorum, Sclerotinia minor, Sclerotinia trifoliorum, Zymoseptoria tritici.

The mixtures and the agrochemical compositions thereof, respectively, are particularly suitable for controlling the following phytophathogenic fungi, Alternaria solanum, Botrytis cinerea, Venturia inaequalis, Leptosphaeria maculans, Leptosphaeria nodorum, Fusarium oxysporum, Fusarium graminearum, Rhizoctonia solani, Phytophthora infestans, Pythium ultimum, Colletotrichum orbiculare, Pyricularia oryzae, Sclerotinia sclerotiorum and Zymoseptoria tritici.

The mixtures and the agrochemical compositions thereof, respectively, are particularly suitable for controlling the following phytophathogenic fungi, Alternaria solanum, Botrytis cinerea, Leptosphaeria nodorum, Phytophthora infestans, Colletotrichum orbiculare, Pyricularia oryzae, Sclerotinia sclerotiorum and Zymoseptoria tritici.

Accordingly, the invention comprises also a method of suppressing or preventing fungal infection of a plant, wherein the fungi, their habitat or the materials or plants to be protected against fungal attack, or the soil or plant propagation material are treated with an effective amount of a Paenibacillus sp. strain comprising
a) no or a mutant PepR or
b) no or a mutant PepH or
c) no or a mutant PepX or
d) no or a mutant ManC or
e) no or a mutant SacB, or
f) a combination of at least two of a), b), c), d), e) or f)
   wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB, and
wherein the Paenibacillus sp. strain comprising the respective wildtype PepR, PepH, PepX, ManC or SacB and used for comparison is of the same Paenibacillus species than the strain comprising no PepR, PepH, PepX, ManC or SacB or a mutant thereof.

Paenibacillus strains produce plant growth producing substances, such as indole acetic acid.
Accordingly, the strains) no or a mutant PepR or
b) no or a mutant PepH or
c) no or a mutant PepX or
d) no or a mutant ManC or
e) no or a mutant SacB, or
f) a combination of at least two of a), b), c), d), e) or f)
   wherein
the missing PepR or the mutant PepR results in a decreased viscosity of a liquid culture of the Paenibacillus sp. strain in comparison to a liquid culture of a Paenibacillus sp. strain comprising a wildtype PepR and
the missing PepH or the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the missing ManC or the mutant ManC results in a decreased viscosity of a liquid culture of the Paenibacillus sp. strain in comparison to a liquid culture of a Paenibacillus sp. strain comprising a wildtype ManC and
the missing SacB or the mutant SacB results in a decreased viscosity of a liquid culture of the Paenibacillus sp. strain in comparison to a liquid culture of a Paenibacillus sp. strain comprising no SacB, and
wherein the Paenibacillus sp. strain comprising the respective wildtype PepR, PepH, PepX, ManC or SacB and used for comparison is of the same Paenibacillus species than the strain comprising no PepR, PepH, PepX, ManC or SacB or a mutant thereof,
   can also be used in a method to enhance the growth of plants by plants, seedlings, seeds or the soil close to the plant or seed is treated with these Paenibacillus strains.

Paenibacillus strains are also known to solubilize plant nutrients. Accordingly, the strains disclosed herein can also be used to mobilize plant nutrients provided by organic or inorganic fertilizer.

In another aspect the invention comprises also methods for production of a valuable product via fermentation of a Paenibacillus sp. strain comprising a) no or a mutant PepR or b) no or a mutant PepH or c) no or a mutant PepX, or d) no or a mutant SacB, or a combination of at least two of a), b), c) or d).

A valuable product can be any product of commercial value, which can be produced via fermentation of Paenibacilli. Non-limiting examples for such products are enzymes used in industrial applications, like in food or feed processing, detergents or chemical synthesis. Examples of such enzymes are phytases, chitinases, proteases, mannanases, xylanases, cellulases, laccases or lipases. These enzymes may be enzymes which are also expressed by wildtype Paenibacillus strains or which are heterologous to the respective Paenibacillus strain and expressed from transgenes. Other examples are proteins (without enzymatic activity), e.g. for use as animal protein replacement, such as milk or meat alternatives.

Further examples of valuable products are secondary metabolites, like vitamins, or other chemical substances of commercial value, like 2,3-Butanediol, lactic acid or acetoin. In case the valuable product is 2,3-Butanediol, lactic acid or acetoin the Paenibacillus sp. strain comprises preferably a mutant or wildtype PepH and or a mutant or wildtype SacB.

Additional examples of valuable products are antimicrobial compounds, like polymyxins, octapeptins, polypeptins, pelgipeptins, fusaricidins or lantibiotics.

Additional examples of valuable products are EPS produced by a Paenibacillus sp. strain comprising a) no or a mutant PepR or b) no or a mutant PepH, c) no or a mutant PepX or d) no or a mutant SacB, or a combination of at least two of a), b), c) or d), wherein the composition of the EPS, differs from a wildtype strain comprising a wildtype PepR, PepH, PepX and SacB.

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

### EXAMPLES

### Example 1 Mutant Generation

A list of strains used for targeted integration of point mutations by CRISPR Cas9 in P. polymyxa is shown in Table 1. Plasmid cloning and multiplication were performed in either E. coli DH5α from NEB (New England Biolabs, USA). Transformation of P. polymyxa was performed by conjugation mediated by E. coli S17-1 (DSMZ). The strains were grown in LB or TSB media. For plate media, 1.5% agar was used. Whenever necessary, the media was supplemented with 50 μg/ml neomycin and/or 20 μg/L polymyxin. P. polymyxa was grown at 30° C. and 250 rpm while E. coli at 37° C. and 250 rpm.

TABLE 1
List of strains used for CRISPR Cas9 mediated construction of targeted
point-mutations in P. polymyxa DSM365

| Strain | Description | Reference | Use |
|---|---|---|---|
| E. coli DH5α | F− ϕ80lacZΔM15 Δ | NEB | High copy plasmid |
| | (lacZYA-argF) 169 recA1 | | amplification of |
| | endA1 hsd R17 (rK−, K+) | | pCasPP before |
| | phoA supE44 λ− thi-1 | | transformation in |
| | gyrA96 relA1 | | S17-1 |
| E. coli S17-1 | recA pro hsdR | ATCC 47055 | Conjugation strain: |
| | RP42Tc::Mu-Km::Tn7 | (DSM 9079) | donor |
| | integrated into the | | |
| | chromosome | | |
| P. polymyxa | Wild type | BASF/DSMZ | Target strain for |
| LU17007 | | 26970 | modification |

Lu17007 had been isolated from crop acreage in Germany and deposited under the Budapest Treaty with the Oeutsche Sammlung von Mikroorganismen und Zellkulturen (DSMZ) under Accession No. DSM 26970 on Feb. 20, 2013

Conjugation was performed between P. polymyxa (recipient strain) and E. coli S17-1 harboring the plasmid of interest (donor strain) according to the CRISPR Cas9 procedure described in Rütering M, Cress B F, Schilling M, Rühmann B, Koffas M A G, Sieber V, Schmid J. Tailor-made exopolysaccharides-CRISPR-Cas9 mediated genome editing in Paenibacillus polymyxa. Synth Biol (Oxf). 2017 Dec. 21; 2 (1):ysx007. doi: 10.1093/synbio/ysx007. Confirmation of the correct conjugants was performed by colony PCR and sequencing of DNA fragments. Plasmid curing was performed by 1:100 subculturing of the positive mutant in LB liquid media at 37 C.

Modifications or knockouts of the pepR and sacB gene were achieved by the CRISPR-Cas9 mediated system established by Rütering M, Cress B F, Schilling M, Rühmann B, Koffas M A G, Sieber V, Schmid J. Tailor-made exopolysaccharides-CRISPR-Cas9 mediated genome editing in Paenibacillus polymyxa. Synth Biol (Oxf). 2017 Dec. 21; 2 (1):ysx007. doi: 10.1093/synbio/ysx007. Selected gRNA sequences were chosen based on their closest proximity to the targeted positions within the pepR and sacB gene. As homologues flanks, 1000 bp of the flanking genes from pepR or sacB, respectively, were used. Using the pCasPP vector system and 1000 the following mutations were generated (table 2):

TABLE 2
List of mutant strains and associated spacer sequences used for genome
editing by CRISPR Cas9. SNP = single nucleotide polymorphism,
nt = nucleotide.

| strain | gene | Modification | spacer sequence 5′→3′ |
|---|---|---|---|
| LU17007 | pepR | Full knockout of pepR | GCTGCTGTATTTCTTCCATA |
| LU17007 | sacB | Point mutation: G232S | TGAGCTGGCGAACGGCGCAC |
| LU17007 | sacB | Full knockout of sacB | TGGCCGTTACAAAATGCCGA |

### Example 2 Fermentation Conditions and Mutant Characterization

Characterization of mutants was carried out in 211 bioreactors (Techfors, Infors), filled with 121 exopolysaccharide production medium adapted from Rtering M, Cress F, Schilling M, Ruhmann B, Koffas MAG, Sieber V, Schmid J. Tailor-made exopolysaccharides-CRISPR-Cas9 mediated genome editing in Paenibacillus polymyxa. Synth Biol (Oxf). 2017 Dec. 21; 2 (1):ysx007. doi: 10.1093/synbio/ysxOO7. The composition of the fermentation medium is listed in Table 3.

Table 3: Composition of the exopolysaccharide production medium with the specification for storage (room temperature (RT) or 4° C.) and sterilization method (sterile-filtered/autoclaved, s/a) of the stock solution.

TABLE 3

| | | Concentration in the |
|---|---|---|
| Stock solution | Component | medium [g/l] |
| Main solution | Potassium dihydrogen phos- | 1.67 |
| (RT, a) | phate | |
| | Magnesium sulfate | 1.33 |
| | heptahydrate | |
| | Calcium chloride dihydrate | 0.05 |
| | Peptone from soy | 5.0 |
| Sugar (RT, a) | Glucose monohydrate | 30 |
| Vitamin solution | Thiamin hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxin hydrochloride | 0.005 |
| | Vitamin B12 | 0.00005 |
| | Lipoic acid | 0.00005 |
| Trace element solution | Manganese sulfate mono- | 0.013 |
| (4° C., s) | hydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Fermentation took place at 30° C. for 40 h, pH was set to 6.8 and adjusted with H3PO4 (25%) and NaOH (1M). As preculture, all mutants were grown for 24 h in 1 L shake flasks with baffles containing 100 ml of modified TSB medium (30 g/L TSB from Becton Dickenson Art.Nr.211825, 3 g/L yeast extract, 20.9 g/L MOPS, 10 g/L glucose) at 33° C. and 150 rpm/2.5 cm throw.

In the bioreactor, target dissolved oxygen level was set a ≥30% in a stirrer-gas flow cascade. To prevent sheering of the exopolysaccharides produced, agitation was limited to 300-600 rpm while using a stirrer setup consisting of two propellers and one Rushton, the latter was placed near the agitator shaft. To maintain oxygen supply, aeration was performed at 5-30 l/min at 0.5 bar pressure. Struktol J673 (Schill+Seilacher “Struktol” GmbH, Germany) was used as antifoam agent. Culture samples were taken every 4 h for rheological viscosity analyses and further offline analytics.

### Example 3 Rheological Analyses of Culture Broth Viscosity

Rheological analysis of broth viscosity was conducted every 4 h during the fermentation using an Anton Paar MCR302 rheometer with double slid geometry (Measuring Cup: C-DG26.7/SS/Air, temperature: 30° C., sample volume: 5 ml of whole culture broth). The samples were preconditioned in a pre-shear experiment at constant shear rate of 10 s−1 for 100 s. 10 data points were recorded every 10 s. After preconditioning, viscosity was measured as a function of the shear rate. Therefore, the shear rate was logarithmically increased from 1 s−1 to 100 s−1 while logging a total of 25 data points. Culture broth viscosity measured over the course of time of the fermentation is depicted in FIG. 1.

1. A Paenibacillus sp. strain comprising
a) no or a mutant PepR or
b) a mutant PepH, or
c) no or a mutant PepX, or
d) a mutant ManC, or
e) a mutant SacB, or
f) a combination of at least two of a), b), c), d) or e),
wherein
the missing PepR or the mutant PepR results in a decreased viscosity of a liquid culture of the Paenibacillus sp. strain in comparison to a liquid culture of a Paenibacillus sp. strain comprising a wildtype PepR and
the mutant PepH results in a decreased viscosity of a liquid culture of the Paenibacillus sp. strain in comparison to a liquid culture of a Paenibacillus sp. strain comprising a wildtype PepH and
the missing PepX or the mutant PepX results in a decreased viscosity of a liquid culture of the Paenibacillus sp. strain in comparison to a liquid culture of a Paenibacillus sp. strain comprising a wildtype PepX and
the mutant ManC results in a decreased viscosity of a liquid culture of the Paenibacillus sp. strain in comparison to a liquid culture of a Paenibacillus sp. strain comprising a wildtype ManC or in comparison to a liquid culture of a Paenibacillus sp. strain comprising no ManC and
the mutant SacB results in a decreased viscosity of a liquid culture of the Paenibacillus sp. strain in comparison to a liquid culture of a Paenibacillus sp. strain comprising no SacB, and
wherein the Paenibacillus sp. strain comprising the respective wildtype PepR, PepH, PepX, ManC or SacB and used for comparison is of the same Paenibacillus species than the comprising no PepR, PepH, PepX, ManC or SacB or a mutant thereof.

2. A Paenibacillus sp. strain as claimed in claim 1, wherein
a) the wildtype PepR has an amino acid sequence which is at least 80%, 90%, 91%, 92%, 93%, 94%, 95%, 96%, 97%, 98%, 99% or 100% identical to Seq ID No. 1 and the mutant PepR comprises an amino acid, which differs from the amino acid at the same place of the amino acid which is 100% conserved in the alignment shown in FIG. 2, or wherein
b) the wildtype PepH has an amino acid sequence which is at least 80%, 90%, 91%, 92%, 93%, 94%, 95%, 96%, 97%, 98%, 99% or 100% identical to Seq ID No. 8, and the mutant PepH comprises an amino acid, which differs from the amino acid at the same place of the amino acid which is 100% conserved in the alignment shown in FIG. 3, or wherein
c) the wildtype PepX has an amino acid sequence which is at least 80%, 90%, 91%, 92%, 93%, 94%, 95%, 96%, 97%, 98%, 99% or 100% identical to Seq ID No. 15, and the mutant PepX comprises an amino acid, which differs from the amino acid at the same place of the amino acid which is 100% conserved in the alignment shown in FIG. 4, or wherein
d) the wildtype ManC has an amino acid sequence which is at least 80%, 90%, 91%, 92%, 93%, 94%, 95%, 96%, 97%, 98%, 99% or 100% identical to Seq ID No. 22, and the mutant ManC comprises an amino acid, which differs from the amino acid at the same place of the amino acid which is 100% conserved in the alignment shown in FIG. 5, or wherein
e) the wildtype SacB has an amino acid sequence which is at least 80%, 90%, 91%, 92%, 93%, 94%, 95%, 96%, 97%, 98%, 99% or 100% identical to Seq ID No. 1 and the mutant SacB comprises an amino acid, which differs from the amino acid at the same place of the amino acid which is 100% conserved in the alignment shown in FIG. 6.

3. A Paenibacillus sp. strain as claimed in claim 1, comprising
a) no or a mutant PepR, or comprising
b) a mutated PepH, or comprising
c) no or a mutant PepX, or comprising
d) a mutated ManC, or comprising
e) a truncated SacB or a SacB having a G323S mutation.

4. A Paenibacillus sp. strain as claimed in claim 1, comprising no PepR.

5. A Paenibacillus sp. strain as claimed in claim 1, comprising no PepX.

6. A Paenibacillus sp. strain as claimed in claim 1, wherein the species of Paenibacillus is selected from Paenibacillus polymyxa, Paenibacillus jamilae, Paenibacillus ottowii, Paenibacillus terrae and Paenibacillus kribensis.

7. A Paenibacillus sp. strain as claimed in claim 1, wherein the parent strain of the Paenibacillus sp. strain is selected form the group of strains comprising P. polymyxa strain DSM365, P. polymyxa strain PKB1, P. polymyxa strain JB05-01-1, P. polymyxa strain AC-1, P. polymyxa strain HY96-2, Paenibacillus sp. Aloe-11, Paenibacillus sp. strains NRRL B-50972, NRRL B-67129, NRRL B-67304, NRRL B-67306 and NRRL B-67615, NRRL B-50374, NRRL B-67721, NRRL B-67723, NRRL B67724, P. polymyxa strain VMC10/96, Paenibacillus sp. strain 10.6D, Paenibacillus sp. strain 9.4E, Paenibacillus sp. strains Lu16774, Lu17007 and Lu17015, Paenibacillus. polymyxa strain M1, Paenibacillus. polymyxa strain SC2, Paenibacillus. polymyxa strain Sb3-1 and Paenibacillus. polymyxa strain E681.

8. An agricultural composition comprising a Paenibacillus sp. strain as claimed in claim 1.

9. A plant propagation material comprising a Paenibacillus sp. strain as claimed in claim 1 or coated with an agricultural composition.

10. A method of suppressing or preventing fungal infection of a plant, wherein the fungi, their habitat or the materials or plants to be protected against fungal attack, or the soil or plant propagation material are treated with an effective amount of a Paenibacillus sp. strain as claimed in claim 1.

11. A method of production of a valuable product via fermentation comprising:
1) culturing a Paenibacillus sp. strain as claimed in claim 1 in a culture medium under conditions in which the valuable product is produced and
2) harvesting the valuable product.

12. A method of production of a valuable product as claimed in claim 11, wherein the valuable product is
a) an enzyme or a protein or
b) a metabolite of Paenibacillus or
c) 2,3-Butanediol, lactic acid or acetoin, or
d) EPS produced by this strain.

13. (canceled)

14. The Paenibacillus sp. strain as claimed of claim 1, comprising
a) no or a mutant PepR comprising a PepR having a W224* or S393F mutation or comprising
b) a mutated PepH comprising a PepH having a E96K or E163K or E96K and a E163K mutation, or comprising
c) no or a mutant PepX comprising a truncated PepX or comprising
d) a mutated ManC comprising a ManC having a P90S, a E340K, or a G433D mutation, or comprising
e) a truncated SacB or a SacB having a G323S mutation.


SEQUENCE LISTING (38 sequences)

SEQ ID NO: 1 - protein, 447 aa, Paenibacillus polymyxa
  REGION 1..447: note = PepR Flippase
MTLVKKRAKPRRSLLVNTSWLFGDKMIRMVFGLAVSIIMARVLGPEELGKWNYAESFFGMFLIFTTLGFDSILVRDLVKHPKDEHELLGTAVLMKLLGTLVAIVLSYSFISLLRPEDSSVRLINLILATASVFQLFDIIDYWFRAQMLSKYTVVAKNTAFVLGSTVKIILLLSGIPIWAVALCAHGEFLLGSLLLLYFFHKEGRRVNKWTFRFTTARRIMNHSWPLILSSSAVYVQARIDQVIIGELLGNEAVGQYSVALRLIEVLGFIPVVLTTAYAPVVTRSKQKGSDEYRRTLSNVYRLMFICFLVTSVPLFFLSQWVVVVLYGREFAEAGSLLSLFAIRLFFTNFSVAKSLFITNENLFKYTLLTSVIGAVTNIAFNYALIPWLGVRGSLVATILSFTISVFLMDLLFKEVKANLGWMMKSILTFWHVHITVPKVGENNHDTD

SEQ ID NO: 2 - protein, 447 aa, Paenibacillus ottowii
  REGION 1..447: note = PepR Flippase
MTLVKKRVKPRRSLLVNTSWLFGDKMIRMVFGLAVSIIMARVLGPEELGKWNYAESFFGMFLIFTTLGFDSILVRDLVKDPKDEHELLGTAVLLKLLGTLVAIVLSYSFISLLRPEDSSVRLINLILATASVFQLFDIIDYWFRAQMLSKYTVVAKNTAFVLGSTAKIVLLLSGTPIWTVALCAHGEFLLGSLLLLYFFHKEGRRVNKWTFRFTTARRIMDHSWPLILSSSAVYVQARIDQVIIGEMLGNEAVGQYSVALRLIEVLGFIPVVLTTAYAPVVTRSKQTGSDEYRRTLSNVYRLMFICFLVTSVPLFFLSQWVVVVLYGREFTEAGSLLSLFAIRLFFTNFSVAKSLFITNENLFKYTLLTSVIGAVTNIAFNYALIPWLGVRGSLVATILSFTISVFLMDLLFKEVKANLGWMMKSILTFWHVHITVPKVGENNHDTD

SEQ ID NO: 3 - protein, 447 aa, Paenibacillus polymyxa
MTLVEKRAKPRRSLLVNTSWLFGDKMIRMVFGLAVSIIMARVLGPEELGKWNYAESFFGMFLIFTTLGFDSILVRDLVKDPKDEHELLGTAVLLKLLGTLIAIVLSYSFISLLRPEDSSVRLINLILATASVFQLFDIIDYWFRAQMLSKYTVVAKNTAFVLGSTVKIILLLSGVPIWMVALCAHGEFLLGSLLLLYFFRKEGRRVNQWTFRLATARRIMNHSWPLILSSSAVYVQARIDQVIIGELLGNAAVGQYSVALRLIEVLGFIPVVLTTAYAPVVTRSKQKGSDEYRQTLSKVYRLMFICFLVTSVPLFFLSQWVVVVLYGREFTEAGSLLSLFAIRLFFTNFSVAKSLFITNENLFRYTLLTSVVGAVTNIAFNYALIPLLGVRGSLVATILSFTISVFLLDLLFKEVKSNLGWMMKSILTFWRVHITVPKVGENNHDTH

SEQ ID NO: 4 - protein, 447 aa, Paenibacillus polymyxa
MTLVKKRAKPRRSLLVNTSWLFGDKMIRMVFGLAVSIIMARVLGPEELGKWNYAESFFGMFLIFTTLGFDSILVRDLVKDPKDEHELLGTAVLLKLVGTLLAIVLSYSFISLLRPEDSSVRLINLILATASVFQLFDIIDYWFRAQMLSKYTVVAKNTAFVLGSIIKIILLLSGVPIWVVALCAHGEFLLGSLLLLYFFHKEGRRVNRWTFRLNTARRIMNHSWPLILSSSAVYVQARIDQVIIGELLGNAAVGQYSVALRLIEVLGFIPVVLTTAYAPVVTRSKQKGSDEYRQTLSTVYRLMFICFLVTSVPLFFLSQWVVVVLYGKEFTEAGSLLSLFAIRLFFTNFSVAKSLFITNENLFRYTLLTSVVGAVTNIAFNYMLIPLLGVRGSLVATILSFTISVFLLDLLFKEVKSNLGWMMKSILTFWRVHITVPKVGENNHDTH

SEQ ID NO: 5 - protein, 447 aa, Paenibacillus terrae
  source 1..447: note = Paenibacillus terrae HPL-003
MTLVKNRAKPRRSLLVNTSWLFGDKMIRMVFGLAVSIIMARVLGPEELGKWNYAESFFGMFLIFTTLGFDSILVRDLVKDPKDEQELLGTAILLKLAGTLVAIVLSCSFISLLRPEDSSVRLINLILATASVFQLFDIIDYWFRAQMLSKYTVIAKNTAFVLSSTVKIILLLSGVPIWVVALCAHGEFLLGSLILLYFFRKEGRRMHKWTFSLTTAQRIMNHSWPLILSSSAVYVQARIDQVIIGEMLGDAAVGQYSVALRLIEVLGFIPVVLTTAYAPVVTRSKMKGSDEYRQTLSNVYRLMFICFLVTSIPLFFLSQWVVVVLYGREFTEAGSLLSLFAIRLFFTNFSVAKSLFITNENLFKYTLLTSIVGAVTNVALNYALIPLLGVRGSLVATILSFTISVFLLDLLFKEVKANLGWMMKSILTFWRVHITVPKVGENNHDTH

SEQ ID NO: 6 - protein, 447 aa, Paenibacillus kribbensis
MTLVKNRVKPRRSLLVNTSWLFGDKMIRMVFGLAVSIIMARVLGPEELGKWNYAESFFGMFLIFTTLGFDSILVRDLVKDPQDEHELLGTAVLLKLSGTLVAIVLSYSFISLLRPEDSSVRLINLILATASVFQLFDIIDYWFRAQMLSKYTVIAKNTAFVLSSTAKIILLLTGVPIWVVALCAHGEFLLGSLLLLYFFRKEGRRMKMWTFRLTIARRIMNHSWPLILSSSAVYVQARIDQVIIGELLGNAAVGQYSVALRLIEVLGFIPVVITTAYAPVVTRSKQKGNDEYKQTLSNVYQLMFICFLVTSVPLFFLSQWVVIVLYGREFSEAGSLLSLFAIRLFFTNFSVAKSLFITNENLFKYTLLTSLVGAITNIAFNYALIPLLGVRGSLVATILSFTISVFLLDVLFKEVKANLGWMMKSILTFWRVHITVPKVGESNHDTH

SEQ ID NO: 7 - protein, 447 aa, Paenibacillus sp.
  source 1..447: note = Paenibacillus sp. Aloe-11
MTLVKNRVKPRRSLLVNTSWLFGDKMIRMVFGLAVSIIMARVLGPEELGKWNYAESFFGMFLIFTTLGFDSILVRDLVKDPQDEHELLGTAVLLKLSGTLVAIALSYSFISLLRPEDSSVRLINLILATASVFQLFDIIDYWFRAQMLSKYTVIAKNTAFVLSSTAKIILLLTGVPIWVVALCAHGEFLLGSLLLLYFFRKEGRRVKMWTFRLNIARRIMNHSWPLILSSSAVYVQARIDQVIIGELLGNAAVGQYSVALRLIEVLGFIPVVITTAYAPVVTRSKQKGNDEYRQTLSNVYRLMFICFLVTSIPLFFLSQWVVIVLYGREFSEAGSLLSLFAIRLFFTNFSVAKSLFITNENLFKYTLLTSLVGAITNIAFNYALIPLLGVRGSLVATILSFTISVFLLDVLFKEVKANLGWMMKSILTFWRVHITVPKVGESNHDTH

SEQ ID NO: 8 - protein, 459 aa, Paenibacillus polymyxa
MQNLQALSAPVRTLWSTVIRFAKSKDNSSAAVKTMIFSMLILVVNMLTGVLTARFLGPTGRGEQTAMVNWSQFLAFCMSFGVPSALIYNAKRKPEETGKLYGLALLLATVFGGMATLIGVFLIPYWLRSFSSSVILFAQCSMMMCPLIAISQINNALLQVRSEYKQYNLFRYLVPLSTLLGLAILILTGTMNPYTSALAYLLPGLPIYIGVTIRMIRLYKPKVKNSWTQFKNLFTYGMGSYGNDLMGQVSTYIDQILIAGLLRPADLGLYAVAVSLARMVNVFSTSIIVVLFPKASGLDKEEAVAITFRAFRVTSTATFLAAVMLMLVAPFVFTLLYGQEFKQALTVFRFLVLEVAISGGTMVLAQQFMALGKPKLVTILQGVGLALVIPLLSILVPRYGLTGAGIAMLSSGILRFVFILCNVKFVLKMKIPRLVISKQDFQWLRSTMSHYIRKKPVNG

SEQ ID NO: 9 - protein, 459 aa, Paenibacillus ottowii
MQNLQALSAPVRTLWSTVIRFAKSKDNSSAAVKTMIFSMLILVVNMLTGVLTARFLGPTGRGEQTAMVNWSQFLAFCMSFGVPSALIYNAKRKPEETGKLYGLALLLATVFGGVATLVGVFLIPYWLRSFSSSVILFAQCSMMMCPLIAISQVNNALLQVRSEYKQYNLFRYLVPLSTLLGLAILILTGTMNPYTSALAYLLPGLPIYIGVTIRMIRLYKPKVKNSWTQFKNLFTYGMGSYGNDLMGQVSTYIDQILIAGLLRPADLGLYAVAVSLARMVNVFSTSIIVVLFPKASGLDKEEAVAITFRAFRITSTATILAALMLMLIAPFVFTLLYGQEFKQALTVFRFLVLEVAISGGTMVLAQQFMALGKPKLVTILQGVGLALVIPLLSILVPRYGLTGAGIAMLSSGILRFVFILCNVKFVLKMKIPRLLISKQDFQWLRSTMSHYIRKKPVNG

SEQ ID NO: 10 - protein, 459 aa, Paenibacillus polymyxa
  source 1..459: note = Paenibacillus polymyxa E681
MQNLQALSAPVRTLWSTVIRFAKSKDNSSAAVKTMIFSMLILVVNMLTGVLTARFLGPTGRGEQTAMVNWSQFLAFCMSFGVPSALIYNAKRKPEETGKLYGLALLLATMFGGLATLVGVFLIPYWLRSFSSSVILFAQCSMMMCPLIAISQINNALLQVRSEYKQYNLFRYLVPLSTLLGLAILILTGNMNPYTSALAYLLPGLPIYIGVTIRMIRLYKPKVKNSWTQFKNLFTYGMGSYGNDLMGQVSTYIDQILIAGLLRPADLGLYAVAVSLARMVNVFSTSIIVVLFPKASGLNKEEAVAITFRAFRVTSTATFLAAVMLMLIAPFVFTLLYGQEFKQALTVFRFLVLEVAISGGTMVLAQQFMALGKPKLVTILQGVGLALVIPLLSILVPKYGLTGAGIAMLSSGILRFIFILCNVKFVLKMKIPRLLISKQDFQWLRSTMSHYIRKKPVNG

SEQ ID NO: 11 - protein, 459 aa, Paenibacillus polymyxa
  source 1..459: note = Paenibacillus polymyxa DSM665
MQNLQALSAPVRTLWSTVIRFAKSKDNSSAAVKTMIFSMLILVVNMLTGVLTARFLGPTGRGEQTAMVNWSQFLAFCMSFGVPSALIYNAKRKPEETGKLYGLALLLATMFGGLATLVGVFLIPYWLRSFSSSVILFAQCSMMMCPLIAISQINNALLQVRSEYKQYNLFRYLVPLSTLLGLGILILTGNMNPYTSALAYLLPGLPIYIGVTIRMIRLYKPKVKNSWTQFKNLFTYGMGSYGNDLMGQVSTYIDQILIAGLLRPADLGLYAVAVSLARMVNVFSTSIIVVLFPKASGLNKEEAVDITFRAFRVTSTATFLAAMMLMLIAPFVFTLLYGQEFKQALTVFRFLVLEVAISGGTMVLAQQFMALGKPKLVTILQGVGLALVIPLLSILVPRYGLTGAGIAMLSSGILRFIFILCNVKFVLKMKIPRLLISKQDFQWLRSTMSHYIRKKPVNG

SEQ ID NO: 12 - protein, 459 aa, Paenibacillus terrae
MQNLQTISAPVRTLWSTVVRFAKSKDSSSAAVKTMIFSMLILVVNMLTGVLTARFLGPTGRGEQTAMVNWSQFLAFCMSFGVPSALIYNAKRNPEETGKLYGLALLLATMFGGLATLIGVFLIPYWLRSFSSSVILFAQCSMMMCPLIAISQINNALLQVRSEYKQYNLFRYLVPLSTLLGLAILILTGHMNPYTSALAYLLPGLPIYIGVTIRMVRLYKPKVKNSWTQFKNLFTYGMGSYGNDLMGQVSTYIDQILIAGLLRPADLGLYAVAVSLARMVNVFSTSIIVVLFPKASGLNKEDAVAITFRAFRVTSTATVLAATMLMLIAPFVFTLLYGPEFKQALTVFRLLVLEVAISGGTMVLAQQFMALGKPKLVTILQGVGLALVIPLLSILVPRYGLTGAGIAMLSSGILRFVFILCNVKFVLKMKIPRLLISKQDIQWLRSTMSHYIRKKPVNG

SEQ ID NO: 13 - protein, 459 aa, Paenibacillus kribbensis
MQNLQTLSAPVRTLWSTVIRFAKSKDNSSAAVKTMIFSMLILVVNMLTGVLTARFLGPTGRGEQTAMVNWSQFLAFCMSFGVPSALIYNAKKKPEETGKLYGLALLLATIFGGLATLVGVFLIPYWLRSFSSSVILFAQCSMMMCPLIAISQINNALLQVRSEYKQYNLFRYLVPLSTLLGLVILILTGHISPYTSALAYLLPGLPIYIGVTIRMIRLYKPKIKNSWTQFKNLFTYGMGSYGNDLMGQVSTYIDQILIAGLLRPADLGLYAVAVSLARMVNVFSTSIIVVLFPKASGLNKEDAVAITFRVFRVTSTVTVLAAVMLTLIAPLVFTLLYGPEFKQALTVFRFLVLEVAISGGTMVLAQQFMALGKPKLVTILQGVGLALVIPLLSILVPKYGLTGAGIAMLSSGILRFVFILCNVKFVLKMKIPRLVISKQDFQWLRSTMAHYIRKKPVNG

SEQ ID NO: 14 - protein, 459 aa, Paenibacillus sp.
  source 1..459: note = Paenibacillus sp. Aloe-11
MQNLQTLSAPVRTLWSTVIRFAKSKDNSSAAVKTMIFSMLILVVNMLTGVLTARFLGPTGRGEQTAMVNWSQFLAFCMSFGVPSALIYNAKKKPEETGKLYGLALLLATMFGGLATLVGVFLIPYWLRSFSSSVILFAQCSMMMCPLIAISQINNALLQVRSEYKQYNLFRYLVPLSTLLGLVILILTGHISPYTSALAYLLPGLPIYIGVTIRMIRLYKPRIKNSWTQFKNLFTYGMGSYGNDLMGQVSTYIDQILIAGLLRPADLGLYAVAVSLARMVNVFSTSIIVVLFPKASGLNKEDAVAITFRVFRVTSTVTVLAALMLTLIAPLVFTLLYGPEFKQALTVFRFLVLEVAISGGTMVLAQQFMALGKPKLVTILQGVGLALVIPLLSILVPKYGLTGAGIAMLSSGILRFVFILCNVKFVLKMKIPRLVISKQDFQWLRSTMAHYIRKKPVNG

SEQ ID NO: 15 - protein, 459 aa, Paenibacillus polymyxa
MSTWTAARKVFTGDSLAKTIMRTSFNNFFVLIVTTLTSILTARMLGVEGKGELAAVLFWPTLIGSVLSFGLPTSLIYNLKKKTGTTEELLALSLWIQLPSSLLAGAVAWICMPLWLNGYGADIIQLSQIYCAAAVPLAVLTALTTALSQGLDRFSVYNGLLFYYPLLNFIGLVTLWLMGLLNVQLAGAVNFAASFLALMWAFLRLRKHMNLRAFRPLTSRQVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLVGACVRLAFSLFSFPMFFNVPLSRIIFDKSDITFVLQTIQKKRRKGELKDAEFASHG

SEQ ID NO: 16 - protein, 459 aa, Paenibacillus ottowii
MSTWTAARKVFTGDSLAKTIMRTSFNNFFVLIVTTLTSILTARMLGVEGKGELAAVLFLPTLIGSVLSFGLPTSLIYNLKKKTGTTEELLALSLWIQLPASLLAGAVAWICMPLWLNGYGADIIQLSQIYCAAAVPLAVLTALTTALSQGLDRFSVYNGLLFYYPLLNFIGLVTLWLMGLLNVQLAGIVNFAASFLALVWAFLRLRKHMNVRAFRPLISRQVLRPYYSYGARVYGMELMGTLSTQTDKIVIVALLSPKAFGLYSVVYALSRVFNVVQNAVTNVTFPKVTGMEHSKIVETVGRAFRISMGAMLIVIVPALFIGRYMLGLLYGPAFLEASLTFYLLSLECIIGGGSWILASAFNALGRPGLVLFRQIVAYAITVGLFFICTPIFGLDGIAIALLVGACVRLAFSLFSFPMFFNVPLSRIIFDKSDITFVLQIIQKKRRKGELKDAEFASHG

SEQ ID NO: 17 - protein, 459 aa, Paenibacillus polymyxa
  source 1..459: note = Paenibacillus polymyxa E681
MSTWTAARKVFTGDSLAKTIMRTSFNNFFVLIVTTLTSILTARMLGVEGKGELAAVLFWPTLIGSVLSFGLPTSLIYNIKKKTGTTEELLTLSLWIQLPASLLAGAVAWICMPLWLNGYGADIIQLSQLYCAAAIPLAVLTALTTALSQGLDRFSVYNGLLFYYPLLNFIGLVTLWLMGLLNVQLAGAVNFAASFLALVWAFLRLRKYMNLRAFRPFTSRQVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLVGACVRLAFSLFSFPMFFNVPLSRIIFDKSDITFVIQTIQKKRRKGELKDAEFASHG

SEQ ID NO: 18 - protein, 459 aa, Paenibacillus polymyxa
  source 1..459: note = Paenibacillus polymyxa DSM665
MSTWTAARKVFTGDSLAKTIMRTSFNNFFVLIVTTLTSILTARMLGVEGKGELAAVLFWPTLIGSVLSFGLPTSLIYNIKKKTGTTEELLALSLWIQLPASLLAGAVAWICMPLWLNGYGADIIQLSQLYCAAAIPLAVLTALTTALSQGLDRFSVYNGLLFYYPLLNFIGLVTLWLMGLLNVQLAGAVNFAASFLALVWAFLRLRKYMNLRAFRPFTSRQVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLVGACVRLAFSLFSFPMFFNVPLSRIIFDKSDITFVIQTIQKKRRKGELKDAEFASHG

SEQ ID NO: 19 - protein, 459 aa, Paenibacillus terrae
MSTWTAARKVFTGDSLAKTIMRTSFNNFFVLIVTTLTSILTARMLGVEGKGELAAVLFWPTLIGSVLSFGLPTSLIYNLKKKTGTTEELLALSLWIQLPASLLAGAVAWICMPLWLNGYGADIVHLSQLYCAAAVPLAVLTALTTALSQGLDRFSVYNGLLFYYPLLNFIGLVTLWLMGLLNVQLAGAVNFAASFLALVWAFLRLRKHMNLRTFRPFTSRQVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLVGACVRLAFSLFSFPMFFDVPLSRIIFDKSDITFVIQTIQKKRRKGELKDAEFASHG

SEQ ID NO: 20 - protein, 459 aa, Paenibacillus kribbensis
MSTWTAARKVFTGDSLAKTIMRTSFNNFFVLIVTTLTSILTARMLGVEGKGELAAVLFWPTLIGSVLSFGLPTSLIYNIKKKTGTTEELLSLSLWIQLPASLLAGAVAWICMPMWLSGYGADIIQLSQLYCAAAIPLAVLTALTTALSQGLDRFSVYNGLLFYYPLLNFIGLVTLWLMGLLNVQLAGTVNFAASLLALMWAFLRLRKHMNLRAFRPFTRREVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLAGACIRLAFSLFSFPMFFNVPLSRIVFDKSDITFVIQTIQKKRRKGELKDAEFASHG

SEQ ID NO: 21 - protein, 459 aa, Paenibacillus sp.
  source 1..459: note = Paenibacillus sp. Aloe-11
MSTWTAARKVFTGDSLAKTIMRTSFNNFFVLIVTTLTSILTARMLGVEGKGELAAVLFWPTLIGSVLSFGLPTSLIYNIKKKTGTTEELLSLSLWIQMPASLLAGAVAWICMPVWLSGYGADIIQLSQLYCAAAIPLAVLTALTTALSQGLDRFSVYNGLLFYYPLLNFIGLVTLWLMGLLNVQLAGTVNFAASLLALMWAFLRLRKHMNLRAFRPFTRREVLRPYYSYGARVYGMELMGTLSTQTDKIVIVALLSPKAFGLYSVVYALSRVFNVVQNAVTNVTFPKVTGMEHSKIVETVGRAFRISMAAMLIVIVPALFIGRYMLGLLYGPAFLEASLTFYLLSLECIIGGGSWILASAFNALGRPGLVLFRQIVAYAITVGLFFICTPIFGLDGIAIALLAGACIRLAFSLFSFPMFFNVPLSRIIFDKSDITFVIQTIQKKRRKGELKDAEFASHG

SEQ ID NO: 22 - protein, 458 aa, Paenibacillus polymyxa
MKLVLLSGGSGKRLWPLSNDSRSKQFLKVLESPEGISESMVQRVWRQLGDNGLSESSFIATGRAQVEMIQSQIGSNTRIIVEPERRDTFPAIALTATYLYSIAGVSPDEIIAILPVDPYVEDAFFASVAQLEHTLQESEGKLALIGVVPSYPSEKYGYIIPKNDATQGSTGYREVSHFQEKPDREQAERLMERNALWNCGVFAFKLSYLLDILASKGLPLNYEEMQKQYASLEKISFDYEVVEKEKDIVVLPYDGFWKDLGTWNTLTEEMSHQQVGRGVVTEDCVNTSLINELDIPVAIIGTNDLIVAASPDGILVTNKAESPRIKEVLKAHDQRPMYEERRWGQYRVVDYVKYDEGNEVLTKRIRVHKGKNISYQLHFKRSEIWTIISGEAEIILNEKLHKVKAGDVVRIPEGTKHSILAVTDVDLIEVQTGSELVEEDIVRFCMDWNEISRHQFIS

SEQ ID NO: 23 - protein, 458 aa, Paenibacillus ottowii
MKLVLLSGGSGKRLWPLSNDSRSKQFLKVLESPEGISESMVQRVWRQLGDNGLSESSFIATGRAQVEMIQSQVGSNTRIIVEPERRDTFPAIALTATYLYSIAGVSPDEIIAILPVDPYVEDAFFASVAQLEHTLQESEGKLALIGVVPSYPSEKYGYIIPKNDAIQGSTGYREVSHFQEKPDREQAERLMERNALWNCGVFAFKLSYLLDILAAKGLPLNYEEMQKQYASLEKISFDYEVVEKEKDIVVLPYNGFWKDLGTWNTLTEEMSHQQVGRGVVTEDCVNTSLINELDIPVAIIGTNDLIVAASPDGILVTNKAESPRIKEVLKAHDQRPMYEERRWGQYRVVDYVKYDEGNEVLTKRIRVHKGKNISYQLHFKRSEIWTIISGEAEIILNEKLHKVKAGDVVRIPEGTKHSILAVTDVDLIEVQTGSELVEEDIVRFCMDWNEISRHQFIS

SEQ ID NO: 24 - protein, 458 aa, Paenibacillus polymyxa
  source 1..458: note = Paenibacillus polymyxa E681
MKLVLLSGGSGKRLWPLSNDSRSKQFLKVLESPEGISESMVQRVWRQLGDNGLSESSFIATGRAQVEMIQSQVGPNTRIIVEPERRDTFPAIALTATYLYSIAGVSPDEIIAILPVDPYVEDAFFASVAQLEHTLQESEGKLALIGVVPSYPSEKYGYIIPKNDATQGSTGYREVSHFQEKPDREQAERLIERNALWNCGVFAFKLSYLLDILASKGLPLNYEEMQKQYASLEKISFDYEVVEKEKDIVVLPYDGFWKDLGTWNTLTEEMSNQQVGRGVVTEDCVNTSLINELDIPVAIIGTNDLIVAASPDGILVTNKAESPRIKEVLKAHDQRPMYEERRWGQYRVVDYVKYDEGNEVLTKRIRVRKGKNISYQLHFKRSEIWTIISGEAEIILNEKLHKVKAGDVVRIPEGTKHSILAVTDVDFIEVQTGSELVEEDIVRFCMDWNEISRHQFIS

SEQ ID NO: 25 - protein, 458 aa, Paenibacillus polymyxa; identical to SEQ ID NO: 22
  source 1..458: note = Paenibacillus polymyxa DSM365

SEQ ID NO: 26 - protein, 458 aa, Paenibacillus terrae
MKLVLLSGGSGKRLWPLSNDSRSKQFLKVLESPEGISESMVQRVWRQLGDNGLSESSFIATGRAQVEMIQSQVGQDTRIIVEPERRDTFPAIALTATYLYSIVGVSPDEIVAILPVDPYVEDAFFATVAQLEHTLQESEGKLALIGVVPSYPSEKYGYIIPKNDTTQGSTGYREVSHFQEKPDREQAERLIERNALWNCGVFAFKLGYLLDILASKGLPLNYEEMQKQYASLEKISFDYEVVEKEKEIVVLPYDGFWKDLGTWNTLTEEMSHQQVGRGVVTEDCVNTSLINELDIPVAIIGTNDLIVAASPDGILVTNKAESPRIKEVLKAHNQRPMYEERRWGHYRVVDYVKYDEGNEVLTKRIRVLQGKNISYQLHFKRSEIWTIISGEAEIILNEKLHKVKAGDVVRIPEGTKHSILAVTDVELIEVQTGSELIEEDIVRFCMDWDDISRHQFIS

SEQ ID NO: 27 - protein, 458 aa, Paenibacillus kribbensis
MKLVLLSGGSGKRLWPLSNDSRSKQFLKVLESPEGISESMVQRVWRQLGDNQLSESSFIATGRAQVEMIQSQVGQDIRIIVEPERRDTFPAIALTATYLYSIAGVSPNEVVAILPVDPYVEDAFFATVAQLEHTLQESEGKLALIGVVPSYPSEKYGYIIPKNDATQGSTGYREVSHFQEKPDREQAERLIERNALWNCGVFAFKLSYLLDILAAKGLPLNYEEMQKQYASLEKISFDYEVVEKEKDIVVLPYDGFWKDLGTWNTLTEEMSHQQVGRGVVTEDCVNTSLINELDIPVAIIGTNDLIVAASPDGILVTNKAESPRIKEVLKAHDQRPMYEERRWGQYRVVDYVKYDEGNEVLTKRIRVRKGKNISYQLHFKRSEIWTIISGEAEIILNEKLHKVKAGDVVRIPEGTKHSILAVTDVDLIEVQTGSELVEEDIVRFCMNWNEISRHQFIS

SEQ ID NO: 28 - protein, 458 aa, Paenibacillus sp.; identical to SEQ ID NO: 27
  source 1..458: note = Paenibacillus sp. Aloe-11

SEQ ID NO: 29 - protein, 499 aa, Paenibacillus polymyxa
MKFNKWFSKAATATVATTLLLGGGVQAFAKGNDEADSKADSAFTQITRSDMLNIFKQQGKEKYEVPSFDASTIKNIPSAIGHDSSGKQIDLDVWDSWPLQNADGTVANYKGYNIVFGLAGDPKRGEDTFIYLFYQKAGDTSLSGWKNAGRVFKDNDKLLSNDPILKNQSEEWSGSATLTSDGQVRLFYTSRQPYDPNNKLYGKQTLSTAQINVSQPDDKTLKVDGVEDLKSIYDGGDGKTYQNVQQSVGIDMDNHTLRDPHYVEDQGHKYIIFEANTGTETGYQGEDSIQNPAYYGGNKNFFKEEQQNLLQSPKKKGAELANGALGIVELNDDYTLKNVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVSSYMTNRGLFPDHKSTFAPSFLLNIKGSKTSVVKNGILEQGQITIDPANDKKEHQNEYGKK

SEQ ID NO: 30 - protein, 499 aa, Paenibacillus ottowii
MKFNKWFSKAATATVATTLLLGGGVQAFANGNDKADSTADSAFTQITRSDMLNIFKQQGKEKYEVPSFDASTIKNIPSAIGHDSSGKQIDLDVWDSWPLQNADGTVANYKGYNIVFGLAGDPKRGEDTFIYLFYQKAGNTSLSGWKNAGRVFKDNDKLLSNDPILKNQSEEWSGSATLTSDGHVRLFYTSRQPYDPNNKLYGKQTLSTAQINVSQPDGETLKVDGVEDLKSIYDGGDGKTYQNVQQSVGIDMDNHTLRDPHYVEDQGHKYIIFEANTGTETGYQGEDSIQNPAYYGGNKKFFTEEQQNLLQSPKKKGAELANGALGIVELNDNFTLKNVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVSSYMTNRGLFPDHKSTFAPSFLLNIKGSKTSVVKNGILAQGQITIDPANDKKDNQNEYGKK

SEQ ID NO: 31 - protein, 499 aa, Paenibacillus polymyxa
  source 1..499: note = Paenibacillus polymyxa E681
MKFNKWFSKAATATVATTLLLGGGVQAFAEENDASDLKTDNAFTQITRNDMLNIFKQQGKEKYEVPSFDASTIKNIPSAMGRDSSGKLIDFDVWDSWPLQNADGTVANYKGYNIVFGLAGDPKRGEDTFIYLFYQKAGNTSLSGWKNAGRVFKDNDKLLANDPILKNQSEEWSGSATLTSDGQVRLFYTSRQPYDPNNKLFGKQTLSTAQINVSQPDDKTLKVDGVEDLKSIYDGGDGKTYQNVQQSVGVDMDNHTLRDPHYVEDQGHKYIIFEANTGTETGYQGEDSLQNSAYYGGNKKFFREELSNLQQSPKKKGAELANGALGIVELNDDYTLKNVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVSSYMTNRGLFPDHKSTFAPSFLLNIKGSKTSVVKNGILEQGQITIDPTNDKKESPNEYGKK

SEQ ID NO: 32 - protein, 499 aa, Paenibacillus polymyxa
  source 1..499: note = Paenibacillus polymyxa DSM365
MKFNKWFSKAATATVATTLLLGGGVQAFAEENDASDLKTDNAFTQITRNDMLNIFKQQGKEKYEVPSFDASTIKNIPSAIGRDSSGKLTDFDVWDSWPLQNADGTVANYKGYNIVFGLAGDPKRAEDTFIYLFYQKAGNTSLSGWKNAGRVFKDNDKLLANDPILKNQSEEWSGSATLTSDGQVRLFYTSRQPFEPSNQLYGKQTLSTAQINVSQPDDKTLKVDGVEDLKSIYDGGDGKTYQNVQQSVGIDMDNHTFRDPHYVEDQGHKYIIFEANTGTETGYQGEDSLQNPAYYGGNKKFFTEEQQNLLQSPKKKGAELANGALGIVELNDDYTLKNVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVSSYMTNRGLFPDHKSTFAPSFLLNIKGSKTSVVKNGILEQGQITIDPTNDKKESPNEYGKK

SEQ ID NO: 33 - protein, 499 aa, Paenibacillus terrae
MEINKFFRKATTLTVATTLLLGGGIQAFAEKTDESDLKSDNAFTQITRHDMLNIFKQQGKEKFEVPSFDASTIKNIPSAKGRDSSGKLIDFDVWDSWPLQNADGTVANYKGYNIVFGLAGDPKKAEDTFIYLFYQKAGNTSLSGWKNAGRVFKDNDKLLVNDPILKNQSEEWSGSATLTSDGQVRLFYTSRQPFEPSNQLYGKQTLTTAQINLSQPDDETLKVDGIDDLKPIYDGGDGKTYQNVQQSVGVDMDNHTFRDPHYVEDQGHKYIIFEANTGTETGYQGEDSLQNSAYYGGNNEFFTEELRNLLQSPKKKGAELANGALGIVELNNDYTLKKVMPPLIASNLVTDEIERANVFKMNGLWYLFTSTRGSKVTVDAIGDDDIYMLGYASTSLTGPYKPLNGTGLVLHQDLDRDDITWTYAHFAIPQGKGNNVVVSSYMTNRGLFPDHKSTFAPSFLLNIKGSKTSVVKNGILEQGQITIDPTDDKKEPPYEYGKK

SEQ ID NO: 34 - protein, 499 aa, Paenibacillus sp.
  source 1..499: note = Paenibacillus sp. Aloe-11
MKINKFFRKATTLTVATTLLLGGGIQAFAEKTDDSDLKTDSAFTQITRHDMLNIFKQQGKEKFEVPSFDASTIKNVSSAKGRDGSGKLIDLDVWDSWPLQNADGTVANYKGYNIVFGLAGDPKRAEDTFIYLFYQKAGNTSLNGWKNAGRVFKDNDKLLANDPILKNQAEEWSGSATLTSDGQVRLFYTSRQPFEPSNQLYGKQTLTTAQINLSQPDDETLKVDGVEDLKPIYDGGDGKTYQNVQQSVGVDMDNHTLRDPHYIEDQGRKYIIFEANTGTETGYQGEDSLQNSAYYGGNNEFFTEELRNLLQSPKKKGAELANGALGIVELNNDYTLKKVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVSSYMTNRGLFPDHKSTFAPSFLLNIKGSKTSVVKNGILEQGQITIDPTDDKKEPPYEYGKK

SEQ ID NO: 35 - protein, 499 aa, Paenibacillus kribbensis
MKINKFFRKATTLTVATTLLLGGGIQAFAEKTDDSDLKTDSAFTQITRHDMLNIFKQQGKEKFEVPSFDASTIKNISSAKGRDGSGKLIDLDVWDSWPLQNADGTVANYKGYNIVFGLAGDPKRAEDTFIYLFYQKAGNTSLNGWKNAGRVFKDNDKLLANDPILKNQAEEWSGSATLTSDGQVRLFYTSRQPFEPSNQLYGKQTLTTAQINLSQPDDETLKVDGVEDLKPIYDGGDGKIYQNVQQSVGVDMDNHTLRDPHYIEDQGRKYIIFEANTGTETGYQGEDSLQNSAYYGGNKKFFTEELHNLLQSPKKKGAELANGALGIVELNNDYTLKKVMPPLIASNLVTDEIERANVFKMNGLWYLFTSTRGSKVTVDAIGDDDIYMLGYVSTSLTGPYKPLNGTGLVLHQDLDRDDITWTYAHFAIPQGKGNNVVVTSYMTNRGLFPDHKSTFAPSFLLNIKGSKTSVVKNRILEQGQIAIDPTNDKEEPPYEYGKK

SEQ ID NO: 36 - DNA, 20 bp, synthetic construct
  source 1..20: mol_type = other DNA
  misc_feature 1..20: note = spacer sequence for CRISPR Cas9 genome editing
gctgctgtatttcttccata

SEQ ID NO: 37 - DNA, 20 bp, synthetic construct
  source 1..20: mol_type = other DNA
  misc_feature 1..20: note = spacer sequence for CRISPR Cas9 genome editing
tgagctggcgaacggcgcac

SEQ ID NO: 38 - DNA, 20 bp, synthetic construct
  source 1..20: mol_type = other DNA
  misc_feature 1..20: note = spacer sequence for CRISPR Cas9 genome editing
tggccgttacaaaatgccga